(19) United States
(12) Patent Application Publication
(10) Pub. No.: US 2026/0315584 A1
(43) Pub. Date: Oct. 15, 2026

(54) RECOMBINANT PROTEIN, EXPRESSION CASSETTE, IMMUNOGENIC COMPOSITION AND USE THEREOF

(71) Applicants: Instituto Butantan, Sao Paulo (BR); Institut Pasteur, Paris (BR)

(72) Inventors: Daniel Ladant, Paris (FR); Maryline Davi, Paris (FR); Maria Leonor Sarno de Oliveira, Sao Paulo (BR); Eliane Namie Miyaji, Sao Paulo (BR); Giovanna de Brito Carneiro, Sao Paulo (BR); Julia Tavares de Castro, Sao Paulo (BR)

(21) Appl. No.: 19/479,132

(22) PCT Filed: Apr. 29, 2024
(86) PCT No.: PCT/BR2024/050175

§ 371 Date: Oct. 27, 2025

(30) Foreign Application Priority Data
Apr. 30, 2023 (EP) 23170891.8

(51) Int. Cl. A61K 39/09; A61P 37/04; C07K 14/315; C12N 9/88

(52) U.S. Cl. CPC A61K 39/092; A61P 37/04; C07K 14/3156; C12N 9/88; C12Y 406/01001; C07K 2319/74

(57) ABSTRACT

The present invention relates to a recombinant protein comprising one or more fragments of pneumococcal surface protein A (PspA) and the adenylate cyclase (CyaA) from Bordetella species, especially Bordetella pertussis, wherein said PspA fragments are selected from clades 1 to 4, or a combination of two or more thereof. Additionally, the invention relates to an expression cassette comprising a DNA sequence encoding said recombinant protein, especially a DNA sequence selected from the group consisting of nucleotide sequences as set forth in SEQ ID NOs: 12 to 18 and degenerate sequences thereof that encode a recombinant protein as set forth in SEQ ID NOs: 5 to 11 respectively. Further, an immunogenic composition comprising said recombinant protein or said expression cassette, and additionally a pharmaceutically acceptable carrier and/or adjuvant is disclosed. Finally, the invention relates to the use of said recombinant protein, or said expression cassette, or said immunogenic composition for the manufacture of a vaccine for preventing infections caused by Streptococcus pneumoniae, wherein said vaccine offers broad-spectrum protection against different pneumococcal isolates, regardless of serotypes.

### FIELD OF THE INVENTION

The present invention is inserted in the field of molecular biology and biochemistry, more specifically, in the field of peptides, since it refers to a recombinant protein comprising the adenylate cyclase (CyaA) and at least one fragment of the pneumococcal surface protein A (PspA). The present invention also relates to the expression cassette encoding said recombinant protein, an immunogenic composition comprising said protein or expression cassette, and their use to obtain a broad-coverage vaccine against Streptococcus pneumoniae.

### BACKGROUND OF THE INVENTION AND STATE OF THE ART

Streptococcus pneumoniae is an important human pathogen, causing diseases such as pneumonia, meningitis and sepsis. In 2016, there were 200 million cases and 1 million deaths from pneumococcal pneumonia, considering all age groups, and 340,000 deaths of children under 5 years of age. Conjugated vaccines (PCV10 (GSK) and PCV13 (Wyeth/Pfizer)) are the main tools against pneumococcal diseases.

However, they confer protection only against 10 or 13 serotypes contained in the formulations, among the 100 serotypes of pneumococcus described. Additionally, an increase in invasive diseases caused by serotypes not included in the formulations is observed in vaccinated populations, reducing their effectiveness. Recently, two new vaccines have been licensed, containing 15 (Merck Sharp & Dohme Corp) and 20 (Wyeth/Pfizer) protein-conjugated capsular polysaccharides, in an attempt to expand serotype coverage. However, the eventual increase in diseases caused by serotypes not included in these formulations must be monitored over time.

In order to solve the existing technical problem, the present invention proposes a recombinant protein (CyaA-PspA) comprising the adenylate cyclase (CyaA) from Bordetella species, especially Bordetella pertussis, which is used for presenting at least one fragment of the pneumococcal surface protein A (PspA) antigen.

CyaA has the ability to bind to receptors present on the surface of cells from the immune system, increasing the immune response to associated antigens. Recombinant CyaA-PspA proteins, containing specific PspA fragments, induce high levels of anti-PspA antibodies, which bind to the surface of several pneumococcal isolates, thus showing reactivity independent of capsular serotype.

Therefore, the present invention allows obtaining recombinant proteins for use in the development of a broad-spectrum vaccine against Streptococcus pneumoniae.

Some prior art documents describe the development of vaccines against Streptococcus pneumoniae comprising certain fragments of PspA.

Brazilian patent application No. PI 10037535, filed on Sep. 28, 2010, published on Jan. 22, 2013, in the name of FUNDAçÃO BUTANTAN, entitled: “COMPOSIçÕES IMUNOGÊNICAS SINÉRGICAS BASEADAS EM ANTÍGENOS PROTÉICOS COMBINADOS COM ANTÍGENO CELULAR PERTUSSIS E TOXINAS INATIVADAS” (Synergic immunogenic compositions based on protein antigens combined with pertussis cell antigen and inactivated toxins) describes immunogenic compositions for the prevention of whooping cough and infections caused by Streptococcus pneumoniae, not specifying the use of the CyaA toxin. In contrast, the present invention proposes a recombinant protein composed by the CyaA from Bordetella species, especially Bordetella pertussis, to obtain a vaccine formulation that offers broad-spectrum protection against different pneumococcal isolates.

International patent application No. PCT/IE2004/000140, filed on Oct. 24, 2004 and published under the No WO2005/035557 (A2) on Apr. 21, 2005, in the name of THE PROVOST, FELLOWS AND SCHOLARS OF THE COLLEGE OF THE HOLY AND UNDIVIDED TRINITY OF QUEEN ELIZABETH, NEAR DUBLIN, entitled: “ADENYLATE CYCLASE IN THE TREATMENT AND/OR PROPHYLAXIS OF IMMUNE-MEDICATED DISEASE” refers to CyaA or a derivative or a mutant or a fragment or a variant or a peptide thereof that can be used for the treatment and/or prophylaxis of an inflammatory and/or immune-mediated disorder and/or autoimmune disease to obtain a vaccine formulation against inflammatory disorder, infections and autoimmune diseases. In addition, this document uses CyaA as an adjuvant and there is no physical fusion between the antigens and the CyaA molecule, at variance with the present invention where the PspA is grafted in recombinant CyaA. Differently, the present invention proposes a vaccine formulation against infections caused by Streptococcus pneumoniae, which comprises CyaA-PspA proteins capable of protecting against infections with pneumococci expressing PspA1, PspA2, PspA3, PspA4 or PspA5.

Brazilian patent application No. PI 1013485 A2, filed on Mar. 23, 2010, published on May 4, 2021, in the name of INSTITUTE OF MICROBIOLOGY OF THE ASCR, V.V.I, INSTITUTE OF PHYSIOLOGY OF THE ASCR, V.V.I e INSTITUT PASTEUR, entitled: “POLIPTÍDEO, DERIVADO DE POLIPEPTÍDEO, COMPOSIçÃO FARMACÊUTICA, USO DE UM DERIVADO DE POLIPTÍDEO, E, MÉTODO PARA A PREPARAçÃO DE UM VETOR PROTEINÁCEO” (Polypeptide, polypeptide derivative, pharmaceutical composition, use of a polypeptide derivative, and method for preparing a protein vector) describe polypeptides derived from CyaA used both in the form of a toxin or detoxified protein, for construction of protein expression vectors, for delivering of molecules, in particular polypeptides, especially antigens, into a cell, in particular cells that express CD11b. Differently, the present invention describes a vaccine formulation against infections caused by Streptococcus pneumoniae, comprising recombinant CyaA-PspA proteins capable of protecting against infections with pneumococci expressing PspA1, PspA2, PspA3, PspA4 or PspA5.

North-American patent application No. US 2010/285068, filed on Jun. 27, 2008, published on Nov. 11, 2010, in the name of UNIVERSIDAD DEL PAIS VASCO, entitled: “METHOD FOR THE INTERNALIZATION OF NON-INVASIVE BACTERIA IN EUKARYOTE CELLS” refers to the internalization of non-invasive bacteria into eukaryotic cells for therapeutic and/or prophylactic purposes; particularly the use of CyaA or its functionally equivalent variant, as an inducing agent for the internalization of non-invasive bacteria in eukaryotic cells. In addition, in this document, CyaA is added to the bacteria to supposedly help their internalization into cells. In any case they show no experiment indicating induction of an immune response aided by CyaA against antigens expressed by the bacteria. However, the present invention distances itself from the aforementioned North-American document by describing a specific vaccine formulation against infections caused by Streptococcus pneumoniae, which comprises recombinant CyaAPspA proteins capable of protecting against infection with pneumococci expressing PspA1, PspA2, PspA3, PspA4 or PspA5.

North-American patent application No. US 2015/320851, filed on Mar. 22, 2013, published on Nov. 12, 2015, in the name of OSAKA UNIVERSITY, entitled: “PNEUMOCOCCAL VACCINE CONTAINING PNEUMOCOCCAL SURFACE PROTEIN A” discloses a pneumococcal vaccine containing pneumococcal surface protein A (PspA). Differently, the aforementioned North-American document is silent regarding the use of CyaA from Bordetella species, especially Bordetella pertussis, and therefore distances itself from the present invention, which proposes a vaccine formulation against infections caused by Streptococcus pneumoniae that comprises recombinant CyaA-PspA proteins.

Brazilian patent application No. BR 11 2015 001350 3, filed on Jul. 23, 2013, published on Aug. 1, 2017, in the name of GENTICEL, entitled “POLINUCLEOTÍDEO, POLINUCLEOTÍDEO QUIMÉRICO, USO DO POLINUCLEOTIDEO, VETOR, CULTURA DE CÉLULA, PROTEÍNA DERIVADA DE CYAA, PROTEÍNA QUIMÉRICA, COMPOSIçÃO, E, MÉTODO PARA PRODUZIR UMA PROTEÍNA QUIMÉRICA” (Polynucleotide, chimeric polynucleotide, use of the polynucleotide, vector, cell culture, CyaA derived protein, chimeric protein, composition, and, method for producing a chimeric protein) discloses a chimeric protein comprising (a) the N-terminal part of the CyaA protein from Bordetella (b) a heterologous polypeptide, and (c) the C-terminal part of the CyaA protein from Bordetella for the production of vaccines against the HPV virus. However, the effective response against HPV infection using the HPV E7 antigen inserted in CyaA is a predominantly cellular response and not an antibody response as expected for a vaccine against Streptococcus pneumoniae based on the PspA antigen, as proposed by the present invention.

The PhD dissertation in biotechnology, entitled, “DESENVOLVIMENTO DE VACINAS PROTEICAS CONTRA STREPTOCOCCUS PNEUMONIAE: CARACTERIZAçÃO DOS COMPONENTES ADJUVANTES DA VACINA CELULAR PERTUSSIS E ANÁLISE DE NOVAS COMBINAçÕES VACINAIS” (Development of protein vaccines against Streptococcus pneumoniae: characterization of the adjuvant components of pertussis cell vaccine and analysis of new vaccine combinations), published in São Paulo (2015), in the name of Carolina Salcedo Rivillas, describes a vaccine formulation composed of pneumococcal surface protein A (PspA) using the pertussis whole cell vaccine or Bordetella pertussis proteins as adjuvants. However, unlike the present invention, in this dissertation PspA was only combined (mixed) to CyaA, producing low amounts of anti-PspA antibodies and no protection against pneumococcal challenge. In the present invention, fragments of PspA are genetically inserted at specific sites within CyaA to make recombinant CyaA-PspA fusion proteins. Thus, this dissertation presents results that show that the simple combination of PspA to adenylate cyclase toxin induces intermediate levels of antibodies against PspA but does not confer significant protection against infection by Streptococcus pneumoniae. Differently, the present invention proposes a vaccine formulation against infections caused by Streptococcus pneumoniae comprising CyaA-PspA recombinant proteins.

The article on behalf of Moreno A T, et al., entitled: “IMMUNIZATION OF MICE WITH SINGLE PSPA FRAGMENTS INDUCES ANTIBODIES CAPABLE OF MEDIATING COMPLEMENT DEPOSITION ON DIFFERENT PNEUMOCOCCAL STRAINS AND CROSS-PROTECTION”, published in Clin Vaccine Immunol, in Epub on Jan. 20, 2010, reveals that PspA4 and PspA5 are capable of inducing antibodies with a high degree of cross-reactivity in vitro, which is reflected in the cross-protection of mice. Differently, the present invention proposes a vaccine formulation against infections caused by Streptococcus pneumoniae comprising CyaA-PspA recombinant proteins.

Thus, although vaccines against infections caused by Streptococcus pneumoniae already exist, there is no motivation in the state of the art to obtain recombinant CyaA-PspA proteins to be used in a broad-spectrum vaccine against infections caused by Streptococcus pneumoniae, as proposed by the present invention.

Therefore, based on the cited state of the art, the currently available multivalent conjugate vaccines are composed of purified capsular polysaccharides from different pneumococcal serotypes conjugated to carrier proteins, which induce protection against invasive diseases and colonization of the nasopharynx in children, but their effectiveness is limited to the serotypes included in the formulations (up to 20 serotypes out of 100 described to date) with concerns of potential replacement of commonly infectious strains by serotypes not present in vaccine formulations.

Therefore, the development of new candidate vaccines capable of inducing protection against a broader range of pneumococcal strains is essential.

### SUMMARY OF THE INVENTION

Advantageously, the present invention proposes recombinant CyaA-PspA proteins capable of inducing high levels of anti-PspA antibodies that react with pneumococcal strains expressing PspA1, PspA2, PspA3, PspA4 and/or PspA5.

The recombinant CyaA-PspA proteins are able to protect mice against invasive challenges with pneumococci expressing PspA2, PspA4 or PspA5, and the antibodies induced by CyaA-PspA proteins have broad-spectrum reactivity against different pneumococcal isolates, indicating that they have good potential for the composition of vaccines that confer serotype-independent protection.

As already mentioned, the present invention will provide significant advantages for broad-spectrum protection against different pneumococcal isolates.

In a first aspect, the present invention relates to a recombinant protein comprising one or more fragments of pneumococcal surface protein A (PspA) and adenylate cyclase (CyaA) from Bordetella species, especially Bordetella pertussis, wherein said fragments derive from PspAs of families 1 and 2, preferably fragments from clades 1 to 4, or a combination of two or more thereof.

In a second aspect, the present invention relates to an expression cassette comprising a DNA sequence selected from the group consisting of nucleotide sequences as set forth in SEQ ID NOs: 12 to 18 and degenerate sequences thereof that encode the recombinant proteins as set forth in SEQ ID NOs: 5 to 11.

In a third aspect, the present invention relates to an immunogenic composition comprising said recombinant protein or expression cassette and a carrier, and optionally a pharmaceutically acceptable adjuvant.

In a fourth aspect, the present invention relates to the use of said recombinant protein, or said expression cassette, or said immunogenic composition for the manufacture of a vaccine for preventing infections caused by Streptococcus pneumoniae, that preferably offers broad-spectrum protection against different pneumococcal isolates, regardless of serotypes.

In a fifth aspect, the invention relates to a method for the prevention of Streptococcus pneumoniae infection in a subject in need thereof wherein said method comprises administering an immunogenic composition according to the invention to said subject.

### BRIEF DESCRIPTION OF THE FIGURES

The present invention, along with its further advantages, may be better understood by referring to the attached images and the following description.

FIG. 1 graphically illustrates the recombinant CyaA-PspA proteins, where (A) is a schematic representation of PspAs from clades 2 and 4 and the respective N-terminal and F5 fragments; (B) refer to proteins that were analyzed through SDS-PAGE, using 8% polyacrylamide gel for CyaA-PspA2-F5 and CyaA-PspA4-F5 proteins (left panel) or 15% polyacrylamide gel for PspA2F5 and PspA4-F5 (right panel), wherein PspA2Pro and PspA4Pro were used as references; (C) refer to Anti-PspA2 (left panels) or antiPspA4 (right panels), analyzed by immunoblots, that recognized PspA2-F5 and PspA4-F5, respectively, either alone or expressed in the CyaA system, wherein PspA2Pro and PspA4Pro were used as positive controls, and CyaA-OVA21 was used as a negative control, arrows indicate bands of interest, and “MW” refers to the full range rainbow molecular weight marker (Cytiva, USA).

FIG. 2 graphically represents the induction of anti-PspA antibodies in mice immunized with CyaA-PspA proteins, in which sera from mice were collected after one, two or three immunizations with the recombinant proteins, and analyzed by ELISA to measure anti-PspA2 (A and C) or anti-PspA4 (B and D) IgG. Circles represent individual levels and lines represent the median for each group. Dashed lines indicate the limit of detection.

FIG. 3 graphically represents the induction of balanced IgG1/IgG2a titers by CyaA-PspA proteins, in which sera from mice immunized with three doses were evaluated for the induction of anti-PspA2 (A) or anti-PspA4 (B) IgG1 and IgG2a titers by ELISA. Bars represent the means of each group, with standard deviations. Numbers above the bars indicate the IgG1/IgG2a ratios (means for each group). IgG1/IgG2a ratios were not calculated for groups with sera below the limit of detection (dashed line).

FIG. 4 graphically represents antibodies induced by immunization with CyaA-PspA proteins that are able to bind to pneumococcal surface, in which the sera obtained after the third immunization were pooled for each group and incubated with different pneumococcal strains. Numbers indicate the median fluorescence intensity for each curve. The names of each strain with serotype and PspA clade are indicated in each graph.

FIG. 5 graphically represents sera from mice immunized with CyaA-PspA proteins that induce complement deposition on pneumococcal surface, in which the sera obtained after the third immunization were pooled for each group and incubated with different pneumococcal strains. Numbers indicate the median fluorescence intensity for each curve. The names of each strain with serotype and PspA clade are indicated in each graph.

FIG. 6 graphically represents the immunization with CyaA-PspA that protects mice from challenges with strains that express homologous and heterologous PspAs, in which mice were immunized with two (B) or three doses (A and C) of the recombinant proteins and were challenged with the indicated pneumococcal strains.

FIG. 7 graphically represents immunization with the CyaA-PspA2-F5-PspA4-F5 protein that induces antiPspA antibodies and protection against pneumococcal infection, wherein (A) refers to the schematic representation of the fusion fragment composed by PspA2-F5 and PspA4-F5 expressed in the CyaA platform; (B) refers to CyaA-PspA2-F5-PspA4-F5 which was analyzed through SDS-PAGE using 8% polyacrylamide; and (C and D) refer to sera from mice that were collected after three immunizations with the recombinant proteins. Induction of anti-PspA2 (C) or antiPspA4 (D) IgG was analyzed by ELISA. Circles represent individual levels and lines represent the median for each group. Dashed lines indicate the limit of detection. (E) refer to mice that were challenged with the A66.1 pneumococcal strain.

FIG. 8 graphically represents antibodies induced by immunization with CyaA-PspA-F5 that bind to the surface of different pneumococcal strains, in which the sera obtained after the third immunization were pooled for each group and incubated with strains from different serotypes. Numbers indicate the mean fluorescence intensity for each curve. The names of each strain with serotype and PspA clade are indicated in each graph.

FIG. 9 graphically represents antibodies induced by immunization with CyaA-PspA2-F5-PspA4-F5 that binds to the surface of different pneumococcal strains, in which the sera obtained after the third immunization were pooled for each group and incubated with strains from different serotypes. Numbers indicate the median fluorescence intensity for each curve. The names of each strain with serotype and PspA clade are indicated in each graph.

FIG. 10 graphically represents the sera from mice immunized with CyaA-PspA2-F5-PspA4-F5 that induce complement deposition on the pneumococcal surface, in which the sera obtained after the third immunization were pooled for each group and incubated with different pneumococcal strains. Numbers indicate the median fluorescence intensity for each curve. The names of each strain with serotype and PspA clade are indicated in each graph.

FIG. 11 graphically represents that the CyaA-A2-A4 protein induces high levels of anti-PspA2 and PspA4 antibodies and protects mice against pneumococcal lung colonization and invasive infection with the 3JYP2670 pneumococcal strain (ST3, PspA4). Mice were immunized with three doses of CyaA-A2-A4 or the PCV13 vaccine. Sera were evaluated for the induction of IgG against PspA2 (A), PspA4 (B) or polysaccharide 3 (PS3, C). Colonization was evaluated in BALF (D) or lungs (E), 12 h after the challenge. IgG against PspA4 (F) or PS3 (G) were evaluated in BALF, 12 h after the challenge. Survival was evaluated for 10 days after the challenge (H). Antibody and CFU levels were evaluated by One-way ANOVA, with Tukey post-test for comparison between groups. Survival was evaluated by the Log-Rank survival curve with the Mantel-Cox test for comparisons between groups. ***P<0.001 and ****P<0.0001. Circles represent individual data and lines represent the mean for each group. Dashed lines indicate the limit of detection for each method (A-G).

FIG. 12 graphically represents that antibodies induced by immunization with CyaA-A2 and CyaA-A4 do not bind to the surface of pneumococcal strains expressing PspA1. Sera obtained after the third immunization were pooled for each group and incubated with different pneumococcal strains that express PspA from clade 1. Binding was evaluated by flow cytometry in a FACS Canto II equipment, after incubation of the samples with anti-IgG-FITC. Results were analyzed by FlowJo V10.1 software with 15,000 events recorded. Numbers indicate the mean fluorescence intensity (MFI) for each curve. The names of each strain with serotype and PspA clade are indicated in each graph. Hat antibodies induced by immunization with CyaA-A2 and CyaA-A4 do not bind to the surface of pneumococcal strains expressing PspA1. Sera obtained after the third immunization were pooled for each group and incubated with different pneumococcal strains that express PspA from clade 1. Binding was evaluated by flow cytometry in a FACS Canto II equipment, after incubation of the samples with anti-IgG-FITC. Results were analyzed by FlowJo V10.1 software with 15,000 events recorded. Numbers indicate the mean fluorescence intensity (MFI) for each curve. The names of each strain with serotype and PspA clade are indicated in each graph.

FIG. 13 is a schematic representation of the insertion of A1 and/or A3 fragments in the AC domain of CyaA. (A) In the first position of CyaA, A1 was inserted to generate CyaA-A1 or the A1-A3 fusion was inserted to generate CyaA-A1-A3. (B) In the second position of CyaA-A2-A4 (CyaA molecule containing A2-A4 in the first position), A1 was inserted to generate CyaA-A2-A4/A1 or the A1-A3 fusion was inserted to generate CyaA-A2-A4/A1-A3. (C) A1-A3 insertion in the 3′ terminal of A4 in the CyaA-A2-A4 molecule to generate CyaA-A2-A4-A1-A3, containing all the fragments in fusion in the same position. (D) Electrophoresis on SDS-PAGE of CyaA-PspA proteins after the purification steps. Lanes: (1) CyaA-A1; (2) CyaA-OVA/A1; (3) CyaA-A1-A3; (4) CyaA-OVAA1-A3; (5) CyaA-A2-A4/A1; (6) CyaA-A2-A4/A1-A3; (7) CyaA-A2 A4-A1-A3. Molecular weight ladder: SeeBlue Plus2 Pre-Stained Protein Standard (Thermo). The CyaA-OVA/A1 (CyaA molecule containing the OVA peptide in the first position and A1 at the second position) and the CyaA-OVA/A1-A3 (CyaA molecule containing the OVA peptide in the first position and A1-A3 at the second position) are intermediate antigens that were constructed to help the obtention of the other proteins and were not proposed as vaccine antigens.

FIG. 14 graphically represents the immunization of mice with different CyaA-PspA based formulations induce antibodies that recognize PspAs from several clades. Mice were immunized with three doses of CyaAA2-A4+CyaA-A1, CyaA-A2-A4/A1 or CyaA-A2-A4/A1-A3. Control groups received CyaA-OVA21. Sera were evaluated for the induction of IgG against PspA1 (A), PspA2 (B) PspA3 (C), PspA4 (D) or PspA5 (E). Antibody and CFU levels were evaluated by the One-way ANOVA, with Tukey post-test for comparison between groups. **P<0.01; ***P<0.001 and ****P<0.0001Circles represent individual data and lines represent the mean for each group. Dashed lines indicate the limit of the method.

FIG. 15 graphically represents antibodies induced by immunization with formulations containing CyaAA2-A4 together with A1 and/or A3, bind to the surface of pneumococcal strains expressing PspA1 and PspA3. Sera obtained after the third immunization were pooled for each group and incubated with different pneumococcal strains that express PspAs from clades 1 or 3. Binding was evaluated by flow cytometry in a FACS Canto II equipment, after incubation of the samples with anti-IgG-FITC. Results were analyzed by FlowJo V10.1 software with 15,000 events recorded. Numbers indicate the mean fluorescence intensity for each curve. The names of each strain with serotypes and PspA clades are indicated in each graph.

FIG. 16 graphically represents antibodies induced by immunization with formulations containing CyaAA2-A4 together with A1 and/or A3, bind to the surface of pneumococcal strains expressing PspA2, PspA4 and PspA5. Sera obtained after the third immunization were pooled for each group and incubated with different pneumococcal strains that express PspAs from clades 1 or 3. Binding was evaluated by flow cytometry in a FACS Canto II equipment, after incubation of the samples with anti-IgG-FITC. Results were analyzed by FlowJo V10.1 software with 15,000 events recorded. Numbers indicate the mean fluorescence intensity for each curve. The names of each strain with serotypes and PspA clades are indicated in each graph.

FIG. 17 graphically represents immunization with CyaA-A2-A4+CyaA-A1, CyaA-A2-A4/A1 or CyaA-A2-A4/A1-A3 proteins protects mice against pneumococcal lung colonization with the EF3030 isolate (ST19F, PspA1). Mice were immunized with three doses of the different formulations and were challenged through the nasal route with the EF3030 pneumococcal strain. Colonization was evaluated in the lungs 24 h after the challenge (A). IgG against PspA1 were evaluated in BALFs before (B) and 24 h after the challenge (C) were evaluated in BALF. Antibody and CFU levels were compared by the One-way ANOVA, with Tukey post-test for comparison between groups. ***P<0.001 and ****P<0.0001. Circles represent individual data and lines represent the mean for each group. Dashed lines indicate the limit of detection for each method.

### DETAILED DESCRIPTION OF THE INVENTION

While the present invention may be susceptible to different embodiments, a preferred embodiment is shown in the following detailed discussion, with the understanding that the present embodiment is to be considered as an exemplification of the principles of the invention and is not intended to limit the present invention to what has been described in this specification.

The present invention relates to a recombinant protein (CyaA-PspA) comprising one or more fragments of pneumococcal surface protein A (PspA) and adenylate cyclase (CyaA) from Bordetella species, especially Bordetella pertussis.

Said PspA fragments were selected from clades belonging to PspAs from families 1 and 2, preferably fragments of clades 1 to 4, or a combination of two or more thereof.

PspA is a virulence factor exposed on the surface of all pneumococcal isolates described to date. PspA helps bacteria to evade the immune system by inhibiting the deposition of complement on the pneumococcal surface and by impairing the bactericidal activity of apolactoferrin, a component of the innate immune responses of mucosal surfaces.

PspA performs an essential role in virulence because it interacts with host immune system and helps the bacteria to evade phagocytosis. PspA is classified into three families consisting of six clades, the PspAs from family 1 (clades 1 and 2), family 2 (clades 3, 4 and 5), and family 3 (clade 6).

In one embodiment of the present invention, said recombinant protein comprises fragments that encode the final part of the N-terminal region of PspAs from clades 2 and 4 (PspA2-F5 and PspA4-F5), belonging to families 1 and 2, respectively.

In one embodiment of the present invention, said recombinant protein comprises fragments that encode the final part of the N-terminal region of PspAs from clades 1 and 3 (PspA1-F5-PspA3-F5), belonging to families 1 and 2, respectively.

In one embodiment of the present invention, said recombinant protein comprises fragments that encode the final part of the N-terminal region of PspAs of clades 2, 4 and 1 (PspA2-F5-PspA4-F5-PspA1-F5).

In one embodiment of the present invention, said recombinant protein comprises fragments that encode the final part of the N-terminal region of PspAs of clades 2, 4, 1 and 3 (PspA2-F5-PspA4-F5-PspA1-F5-PspA3-F5).

It is worth mentioning that the final part of the N-terminal region is a variable portion of the molecule, and each initial or final amino acid varies according to the sequence. Thus, the final part of the N-terminal region varies for each molecule, even within a clade.

In one embodiment of the invention, more specifically, said PspAs fragments comprise at least one amino acid sequence selected from the group comprising or consisting of:
PspA2-F5 consists of the amino acid sequence as set forth in SEQ ID NO: 1;
PspA4-F5 consists of the amino acid sequence as set forth in SEQ ID NO: 2;
PspA1-F5 consists of the amino acid sequence as set forth in SEQ ID NO: 3; and
PspA3-F5 consists of the amino acid sequence as set forth in SEQ ID NO: 4.

In another embodiment of the invention, said PspAs fragments comprise at least one amino acid sequence selected from the group comprising or consisting of amino acid sequences having at least 70%, 75%, 80%, 85%, 90%, 95%, 96%, 97%, 98%, or 99%, or more identity with SEQ ID NO: 1-4.

In one embodiment of the invention, said PspAs fragments are encoded by the nucleotide sequences selected from the group comprising or consisting of:
The DNA sequence as set forth in SEQ ID NO: 29 and degenerate sequences thereof that encode the PspA2-F5 as set forth in SEQ ID NO: 1;
The DNA sequence as set forth in SEQ ID NO: 30 and degenerate sequences thereof that encode the PspA4-F5 as set forth in SEQ ID NO: 2;
The DNA sequence as set forth in SEQ ID NO: 31 and degenerate sequences thereof that encode the PspA1-F5 as set forth in SEQ ID NO: 3; and
The DNA sequence as set forth in SEQ ID NO: 32 and degenerate sequences thereof that encode the PspA3-F5 as set forth in SEQ ID NO: 4.

In another embodiment of the invention, said PspAs fragments are encoded by nucleotides sequences selected from the group comprising or consisting of nucleotides sequences having at least 70%, 75%, 80%, 85%, 90%, 95%, 96%, 97%, 98%, or 99%, or more identity with SEQ ID NO: 29-32.

The PspA fragments are cloned into permissive sites of CyaA, and thus recombinant CyaA-PspA proteins are obtained. For this, CyaA was engineered to express the referred fragments of the N-terminal region of PspAs described herein. The adenylate cyclase (CyaA) is from Bordetella species (e.g., Bordetella pertussis, Bordetella parapertussis, Bordetella bronchiseptica, Bordetella hinzii) and particularly from Bordetella pertussis. Bordetella pertussis amino acid sequence and nucleotide sequence are referred to as SEQ ID NO: 33 and 34, respectively.

Bordetella pertussis CyaA is a 1706 residue protein composed of an N-terminal catalytic domain (AC) and a C-terminal RTX-like region, involved in toxin binding to CD11b/CD18 receptors expressed by antigen presenting cells. Upon binding, the adenylate cyclase (AC) domain enters the host cells and catalyzes the production of cyclic adenosine monophosphate (cAMP) subverting cell activity.

CyaA has affinity for CD11b receptors present in cells of the immune system, such as dendritic cells (DCs). The antigens of interest can be genetically or chemically fused to CyaA, generating recombinant proteins that are delivered in vivo to DCs, inducing the production of specific antibodies and activating T-CD4 and T-CD8 lymphocytes. As a result, CyaA enhances the specific immune response to associated antigens.

The protein used is CyaA or a functional fragment thereof. Said fragment may be truncated CyaA, wherein residues at either or both terminal ends are deleted. In particular, residues at the C-terminal end may be deleted to the extent that it does not affect the recognition and binding site for the CD11b/CD18 cell receptor. Alternatively, or in addition, residues may be deleted at the N-terminal end so long as it does not affect the translocation ability of the CyaA fragment. It may also be a fragment obtained after internal deletions of one or more residues of the native CyaA protein. A particular fragmente is one which corresponds to the CyaA protein wherein amino acid residues 225 to 234 have been deleted, thus providing a CyaA fragment containing residues 1 to 224 and 235 to 1706 (when reference is made to the amino sequence of the CyaA protein of Bordetella pertussis). A functional fragment retains the ability of the full-length CyaA to bind cells, especially CD11b/CD18 expressing cells, and eventually to translocate its N-terminal domain into the cytosol of target cells.

In a particular embodiment, the enzymatic activity of the CyaA protein, i.e., its ability to convert ATP into CAMP, is inactivated. Such inactivation may be obtained as a result of genetic inactivation. As an example, genetic inactivation can be obtained as a result of introduction of a dipeptide in a site of the amino acid sequence of CyaA which is part of the catalytic site (for example between 188 and 189). Preferably, the process consists of the insertion of leucine (L) and glutamine (Q) residues between the amino acids aspartate (D) at position 188 and isoleucine (I) at position 189. These insertions abolish the activity of adenylate cyclase. The inactivation in this specification is not limited to insertion of a dipeptide between 188 and 189. Inactivation of enzymatic activity can also be obtained by modifying certain amino acid implicated in catalysis (e.g. Lys58 changed to Gln, Lys65 changed to Gln (Glaser et al, 1989), Asp 188 changed to Asn, Asp 190 changed to Asn, His298 changed to Leu (Glaser et al, 1991) Insertions of antigen fragments between Glycine at position 335 and Glutamine at position 336 also abolishes the adenylate cyclase enzymatic activity (Ladant et al. 1992.

Thus, recombinant proteins were developed by mutating residues important for the enzymatic activity of adenylate cyclase, in the AC domain of CyaA, making the toxin inactive (through the presence of the DLQI sequence in CyaA, as already mentioned). A “permissive site” is a site of the sequence of the CyaA protein where a polypeptide can be inserted without substantially affecting the functional properties of the CyaA protein especially without substantially affecting the targeting of cells, particularly targeting of APC by CyaA, including without substantially affecting the specific binding to the CD11b-CD18 receptor and advantageously without substantially affecting the domains of the protein involved in the process of translocation of the epitope(s) into a target cell. Permissive sites of the Bordetella pertussis adenylate cyclase allowing translocation of CyaA catalytic domain and hence translocation of epitopes inserted into such permissive sites include, but are not limited to, residues 137-138 (Val-Ala), residues 224-225 (Arg-Ala), residues 228 229 (Glu-Ala), residues 235-236 (Arg-Glu), and residues 317 318 (Ser-Ala) (Sebo et al., 1995), residues 107-108 (GlyHis), residues 132-133 (Met-Ala), residues 232-233 (Gly-Leu), residues 319-320 (Thr-Gly), residues 335-336 (Gly-Gln) and residues 336-337 (Gln-Gln). In a particular embodiment, the at least one PspA fragment(s) is (are) inserted between residues 224 and 225 of Bordetella pertussis adenylate cyclase. In another particular embodiment, the at least one PspA fragment is inserted between residues 319 and 320 of Bordetella pertussis adenylate cyclase. In still another particular embodiment, the Bordetella pertussis adenylate cyclase comprises at least two PspA fragments and at least one PspA fragment is inserted between residues 224 and 225 of Bordetella pertussis adenylate cyclase and at least one PspA fragment is inserted between residues 319 and 320 of Bordetella pertussis adenylate cyclase.

In one embodiment of the present invention, the recombinant protein obtained is selected from the group comprising or consisting of the amino acid sequences:
The recombinant protein CyaA-PspA2-F5 consists of the amino acid sequence as set forth in SEQ ID NO: 5;
The recombinant protein CyaA-PspA4-F5 consists of the amino acid sequence as set forth in SEQ ID NO: 6;
The recombinant protein CyaA-PspA2-F5-PspA4-F5 consists of the amino acid sequence as set forth in SEQ ID NO: 7;
The recombinant protein CyaA-PspA1-F5 consists of the amino acid sequence as set forth in SEQ ID NO: 8;
The recombinant protein CyaA-PspA1-F5-PspA3-F5 consists of the amino acid sequence as set forth in SEQ ID NO: 9;
The recombinant protein CyaA-PspA2-F5-PspA4-F5-PspA1-F5 consists of the amino acid sequence as set forth in SEQ ID NO: 10; and
The recombinant protein CyaA-PspA2-F5-PspA4-F5-PspA1-F5-PspA3-F5 consists of the amino acid sequence as set forth in SEQ ID NO: 11.

In another embodiment of the present invention, the recombinant protein obtained is selected from the group comprising or consisting of amino acid sequences having at least 70%, 75%, 80%, 85%, 90%, 95%, 96%, 97%, 98%, or 99%, or more identity with SEQ ID NO: 5-11.

In one embodiment, the recombinant protein of the present invention is encoded by a nucleotide sequence selected from the group comprising or consisting of:
The DNA sequence as set forth in SEQ ID NO: 12 and degenerate sequences thereof encode a recombinant protein CyaA-PspA2-F5 as set forth in SEQ ID NO: 5;
The DNA sequence as set forth in SEQ ID NO: 13 and degenerate sequences thereof encode a recombinant protein CyaA-PspA4-F5 as set forth in SEQ ID NO: 6;
The DNA sequence as set forth in SEQ ID NO: 14 and degenerate sequences thereof encode a recombinant protein CyaA-PspA2-F5-PspA4-F5 as set forth in SEQ ID NO: 7;
The DNA sequence as set forth in SEQ ID NO: 15 and degenerate sequences thereof encode a recombinant protein CyaA-PspA1-F5 as set forth in SEQ ID NO: 8;
The DNA sequence as set forth in SEQ ID NO: 16 and degenerate sequences thereof encode a recombinant protein CyaA-PspA1-F5-PspA3-F5 as set forth in SEQ ID NO: 9;
The DNA sequence as set forth in SEQ ID NO: 17 and degenerate sequences thereof encode a recombinant protein CyaA-PspA2-F5-PspA4-F5-PspA1-F5 as set forth in SEQ ID NO: 10; and
The DNA sequence as set forth in SEQ ID NO: 18 and degenerate sequences thereof encode a recombinant protein CyaA-PspA2-F5-PspA4-F5-PspA1-F5-PspA3-F5 as set forth in SEQ ID NO: 11.

In another embodiment, the recombinant protein of the present invention is encoded by a nucleotide sequence selected from the group comprising or consisting of nucleotide sequences having at least 70%, 75%, 80%, 85%, 90%, 95%, 96%, 97%, 98%, or 99%, or more identity with SEQ ID NO: 5-11.

Additionally, the inventors of the present invention declare that there was access to the Brazilian National Genetic Heritage and/or Associated Traditional Knowledge in the research and technological development that gave rise to the present patent application and inform that the registration number (Sisgen) is A3B0F7E.

Additionally, the present invention relates to an expression cassette comprising the DNA sequence according to the invention especially a DNA sequence selected from the group consisting of nucleotide sequences as set forth in SEQ ID NOs: 12 to 18 and degenerate sequences thereof that encode a recombinant protein as set forth in SEQ ID NOs: 5 to 11.

Additionally, the present invention relates to an immunogenic composition comprising said recombinant protein or said expression cassette, and additionally a pharmaceutically acceptable carrier and/or adjuvant.

Preferably, said immunogenic composition is in the form of a vaccine.

A pharmaceutically acceptable carrier means a non-toxic, inert solid, semi-solid liquid excipient, diluent, formulation adjuvant of any type, or simply a sterile aqueous medium such as saline. Some examples of materials that can serve as pharmaceutically acceptable carriers and/or adjuvants are sugars, such as lactose, glucose and sucrose, starches, such as corn starch and potato starch, cellulose and its derivatives, such as sodium carboxymethylcellulose, ethyl cellulose and cellulose acetate, cyclodextrin; oils, such as peanut oil, cottonseed oil, sunflower oil, sesame oil, olive oil, corn oil and soybean oil; glycols such as propylene glycol, polyols such as glycerin glycol, sorbitol, mannitol and polyethylene; esters, such as ethyl laurate, ethyl oleate, agar; buffering agents such as aluminum hydroxide and magnesium hydroxide; alginic acid; pyrogen-free water; isotonic saline, Ringer's solution; Ethyl alcohol and phosphate buffer solutions, as well as other compatible non-toxic substances used in pharmaceutical formulations.

Thus, a pharmaceutically acceptable carrier and/or adjuvant is any pharmaceutically acceptable carrier or adjuvant known from the state of the art for obtaining a vaccine.

In one embodiment, said immunogenic composition is administered parenterally, which includes subcutaneous, intradermal, and intramuscular routes.

In one embodiment, said immunogenic composition is administered via the mucosal route, preferably the nasal route.

Additionally, the present invention relates to the use of said recombinant protein, or said expression cassette, or said immunogenic compositions for the manufacture of a medicine for preventing infections caused by Streptococcus pneumoniae.

Preferably, said medicine is a vaccine for preventing infections caused by Streptococcus pneumoniae that provides broad-spectrum protection against different pneumococcal isolates, regardless of serotypes.

In particular embodiments, recombinant CyaA-PspA proteins of the present invention are able to induce high levels of anti-PspA antibodies that reacted with pneumococcal strains expressing PspA2, PspA3, PspA4 and PspA5.

In addition, PspA4 fragment of the present invention induces a cross-reaction against PspA5 satisfactorily, without the need to include PspA5 in the immunogenic composition. Thus, PspA4 induces cross-reacting antibodies, also protecting against pneumococcal strains expressing clade 5 PspA.

Thus, the present invention developed a vaccine candidate capable of inducing potent immune responses against PspA in mice, exploiting the adjuvant properties of the Bordetella species, especially Bordetella pertussis, adenylate cyclase (CyaA), and the resulting CyaA-PspA proteins that were used in experiments of immunization in mice.

While the present invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Materials and Methods

### Bacteria:

DNA cloning was performed in E. coli XL1-Blue or DH5a for the CyaA-PspA2-F5 (SEQ ID NO: 5) constructs or the CyaA-PspA4-F5 (SEQ ID NO: 6) construct, respectively. CyaA-PspA-F5 proteins, were expressed in E. coli BLR strain, whereas E. coli BL21 Star pLyS strain was used for the expression of PspA-F5 proteins. Bacteria were grown on LB media (Difco) at 37° C. supplemented with 100 μg/ml ampicillin for plasmid selection.

Streptococcus pneumoniae strains ATCC6303 (serotype 3, PspA5); 3JYP2670 (serotype 3, PspA4); A66.1 (serotype 3, PspA2); D39 (serotype 2, PspA2); M10 (serotype 11A, PspA3); TIGR4 (serotype 4, PspA3) were grown on Tryptic soy agar containing 5% defibrinated sheep blood (blood agar, Laborclin, Brazil) at 37° C. Stocks for animal challenge were prepared in liquid Todd-Hewitt media (Difco) supplemented with 0.5% yeast extract (THY). Bacteria were grown to exponential phase (OD600 nm=0.4), centrifuged and suspended in 1/10 of the initial volume in THY containing 20% glycerol. Stocks were maintained at −80° C. and quantified by plating on blood agar.

### Plasmids and Recombinant Proteins:

The pspA-F5 fragments encode the last part of the N-terminal region of PspAs including the clade-defining region (CDR) of PspA2 (pneumococcal strain Rx1, GenBank Accession number M74122.1) or PspA4 (pneumococcal strain 255/00, GenBank Accession number EF649969). Fragments were amplified by PCR using the pAE-pspARXI plasmid (Vadesilho C F et al., 2014) or the pAE-PspA4Pro plasmid (Darrieux M et al, 2008) as templates.

For the CyaA-PspA constructs, DNA fragments corresponding to pspA2-F5 (SEQ ID NO: 12) and pspA4-F5 (SEQ ID NO:13) were amplified with the following specific primers (the Nhe I and Kpn I restriction sites are in bold cases):

cya-pspA2-F5
Forw
(SEQ ID NO: 19):
5′-GGCGCGTACGCGTAGGCCTTGCTAGCTCTGAATCAGAAGATT
ATGCTAAA 3′,
and
cya-pspA2-F5
Rev
(SEQ ID NO: 20):
5′-CCGTGGCCTCGCTGGCGGCGGTACCTGGAGTTTCTGGAGCTG
GAGCTGG 3′;
or
cya-pspA4-F5 Forw
(SEQ ID NO: 21):
5′ GGCGCGTACGCGTAGGCCTGCTAGCTCAAACGGTGAGCAAG
CTGA 3′;
and
cya-pspA4-F5 Rev
(SEQ ID NO: 22):
5′ CGTGGCCTCGCTGGCGGCGGTACCTGGAGCTGGAGCTGG
TTTTTCT 3′.

The purified DNA fragments were then cloned between the Nhe I and Kpn I sites of the pCACT-E5 vector (Guermonprez P et al, 2000) using the Gibson assembling technique (Karimova G et al, 1998) to generate pCACT-pspA2 F5 and pCACT-pspA4-F5 expression vectors. Correct insertion was verified by nucleotide sequencing that was performed by Eurofins (the nucleotide sequences of the vectors are available upon request). In addition, a plasmid expressing CyaA carrying both pspA2-F5 (SEQ ID NO:1) and pspA4-F5 (SEQ ID NO: 2) fragments was constructed as follows. A DNA fragment encoding pspA2-F5 was amplified from pCACT-pspA2 F5 with primers A2-f51 of SEQ ID NO: 27 (5′-GACCGATTACCTGGCGCGTACGCGCCGTTCTGAATCAGAAGATTAT GCT-3′) and A2-f52 of SEQ ID NO: 28 (5′-TGAGCTAGCAGGCCTACGCGTACCTGGAGTTTCTGGAGCTGG-3′), purified and sub-cloned by Gibson cloning into pCACT-pspA4-F5 linearized by BsiWI (correct insertion was verified by nucleotide sequencing as above). The resulting plasmid pCACT-pspA2-A4-F5, expresses a recombinant CyaA carrying both pspA2-F5 and pspA4-F5 fragments separated by a small peptidic linker (TRRPAS, one letter code for amino acids). Expression and purification of the CyaA-PspA-F5 proteins were performed as previously described (Guermonprez P et al, 2000; Karimova G et al, 1998; and Preville X et al, 2005).

Briefly the expression plasmids were transformed into the E. coli BLR strain (Novagen, Sigma-Aldrich). The transformed cells were grown in LB medium at 37° C. until mid-log phase and expression of the recombinant CyaA-PspA proteins was induced by addition of 0.5 mM IPTG (Isopropyl R-D-1-thiogalactopyranoside). After 2.5 h of additional growth at 37° C., bacteria were collected by centrifugation, resuspended in 20 mM Hepes-Na buffer (pH 7.5) and lysed in a sonicator (Branson Sonifier® 250, Emerson Electric Co, USA). Purification of the recombinant proteins was performed from inclusion bodies solubilized with 8M urea, 20 mM Hepes-Na, pH 7.5. Proteins were purified by an ion exchange chromatography on DEAE-sepharose followed by an hydrophobic chromatography on phenyl-sepharose that included a cleaning step with an isopropanol wash to eliminate endotoxin contamination, as previously described (Preville X et al, 2005).

Protein concentrations were determined by spectrophotometry based on the absorption at 278 nm, using for each molecule the molecular extinction coefficient, computed from the amino acid sequence (http://www.expasy.org/tools/protparam.html).

The CyaA-OVA protein of SEQ ID NO: 25 (wherein the sequence of nucleotides of CyaA-OVA is SEQ ID NO: 26), used as negative control in the immunization experiments, was produced with the same procedures, using the BLR bacteria transformed with the pCACT-E5-CyaA-OVA21 plasmid (Guermonprez P et al, 2000). Construction of the pAE-pspA2 F5, pAE-pspA2Rx1 and pAE-pspA4Pro vectors was previously described (Vadesilho C F et al., 2014; and Darrieux M et al, 2008). For the construction of the pAE-pspA4-F5 plasmid, the fragment was amplified using the pAE-pspA4Pro vector as template and the following primers:

SEQ ID NO: 23:
Forw
5′ GGATCCTCAAACGGTGAGCAAGCTGAAC 3′;
and
SEQ ID NO: 24:
Rev:
5′ AAGCTTTCATGGAGCTGGAGCTGGTTTTTCTGG 3′.

The pspA4-F5 fragment was cloned into the Bam HI and Hind III sites of the pAE vector (Teodorowicz M et al, 2017). Expression of PspA2-F5, PspA4-F5, PspA2Pro and PspA4Pro was induced in BL21 Star pLyS by the addition of 1.2 mM IPTG to the cultures, for 3 h at 37° C. Bacteria were lyzed in a PANDA homogenizer (GEA Niro Soavi, Italy) and proteins were purified through affinity chromatography in HisTrap 5 mL columns (Cyvita, USA) as previously described (Darrieux M et al, 2008).

An additional purification step, for the removal of excess LPS, was performed though treatment with 2% triton X-114, followed by detergent removal with BioBeads SM-2 Resin (Bio-rad Laboratories, USA) (Salcedo-Rivillas C et al, 2014). Purified proteins were analyzed by SDS-PAGE and quantified using the Bio Rad Protein Assay reagent (Bio Rad, USA). Western-blots were performed using polyclonal anti-PspA2 or anti-PspA4 antisera (produced in the laboratory) and Horseradish Peroxidase (HRP)-conjugated anti-mouse IgG (Sigma-Aldrich, USA). Results were evaluated using the Amersham ECL Prime detection reagent (Cyvita) and the Amersham ImageQuant 800 equipment (Cyvita).

### Immunization of Mice and Analysis of Immune Responses:

Female SPF BALB/c mice were used and the protein doses were adjusted for 50 μmol per mice, considering the differences in molecular weight between the CyaA-PspAF5 proteins (around 200 kDa, 10 μg per dose) and the PspA-F5 proteins (11.3 kDa and 0.6 μg per dose for PspA2-F5 and 17.4 kDa and 0.9 μg per dose for PspA4-F5).

For the analysis of the fusion protein, mice were immunized with 10 μg of CyaA-PspA2-F5-PspA4-F5 (SEQ ID NO: 7) or 20 μg of the mixture of CyaA-PspA2-F5 (SEQ ID NO: 5)+CyaA-PspA4-F5 (SEQ ID NO: 6) (10 μg of each protein) as a positive control.

In all experiments, control groups received purified CyaA-OVA21 (10 μg per dose) or saline. Mice (6 per group) received two or three subcutaneous doses at 15-day intervals.

Blood was collected 14 days after the second and third immunizations through the retro orbital plexus, under local anesthesia with 5% proxymetacaine chloride eye drops (Alcon, USA).

Induction of anti-PspA IgG was evaluated by ELISA using plates coated with 1 μg/ml of PspA4Pro or PspA2Pro and serial dilutions of sera prepared from blood samples. Horseradish Peroxidase (HRP) conjugated anti-mouse IgG (Sigma-Aldrich, USA) was used as secondary antibody. For the detection of IgG subtypes, anti-mouse IgG1, anti-mouse IgG2a produced in goat (Southern Biotec, USA) and HRP anti-goat IgG were used (Southern). Reactions were developed using o-Phenylenediamine dihydrochloride as substrate and the absorbances were measured at 492 nm in a spectrophotometer multiskan EX (Thermo Fisher Scientific, USA).

Antibody titers were defined as the reciprocal of the dilution that produced an absorbance of 0.1. Sera were also tested for binding to the surface of different pneumococcal strains, as described in SALCEDO-RIVILLAS C., et al., 2014.

Briefly, bacterial suspensions (10⁸ CFU/mL) were incubated with 5% (V/V) of heat-inactivated sera pooled from each experimental group, for 30 min on ice. Samples were washed once with PBS and incubated with fluorescein isothiocyanate (FITC)-conjugated goat anti-mouse IgG (MP Biomedicals, USA), for 30 minutes on ice.

For the analysis of the induction of complement deposition, samples were incubated with sera from naïve mice as complement source, for 30 min at 37° C., followed by incubation with FITC-conjugated anti-mouse C3 (MP Biomedicals) for 30 min on ice.

Samples were suspended in cytofix (BD Biosciences, USA) and analyzed by flow cytometry in a FACS Canto II equipment (BD Biosciences), with 15,000 gated events recorded. Medians of fluorescence intensity were compared using the Flow Jo 10.1 software.

### Invasive Pneumococcal Challenge:

Twenty-one days after the last immunization, animals were anesthetized through the intraperitoneal route (i.p.) with 100 mg/Kg of ketamine chloride (Ceva, Brazil) and 20 mg/Kg of xylazine chloride (Ceva). S. pneumoniae strains A66.1 (1×10⁶ CFU per mice), 3JYP2670 (3×10⁵ per mice) or ATCC6303 (3×10⁵ per mice) were inoculated through the intranasal route, in 50 μL of saline.

Animals were monitored twice a day, for 10 days, and those that presented signs of the disease with hair erection, compromised posture and reduced activity, were euthanized immediately with a lethal dose of anesthetics (60 mg/Kg of xylazine chloride and 300 mg/kg ketamine chloride, i.p.). Mice that remained until the end of the experiment were healthy and active; no animals died before meeting the criteria for euthanasia.

### Statistical Analysis:

Antibody titers among all groups were compared by One-way ANOVA, and the comparisons between two groups were performed by Tukey's post-test. Animal Survival was analyzed by the Log-rank survival curve using the Mantel-Cox test for comparison between groups. Prism GraphPad 6.0 software was used for the analyzes and P<0.05 was considered significantly different.

### Experimental Results

### Expression of PspA2-F5 and PspA4-F5 in the CyaA System:

In one embodiment of the present invention, fragments of PspAs from family 1 (PspA2) and family 2 (PspA4) were expressed in the CyaA protein, aiming to induce antibodies that react with different pneumococcal isolates.

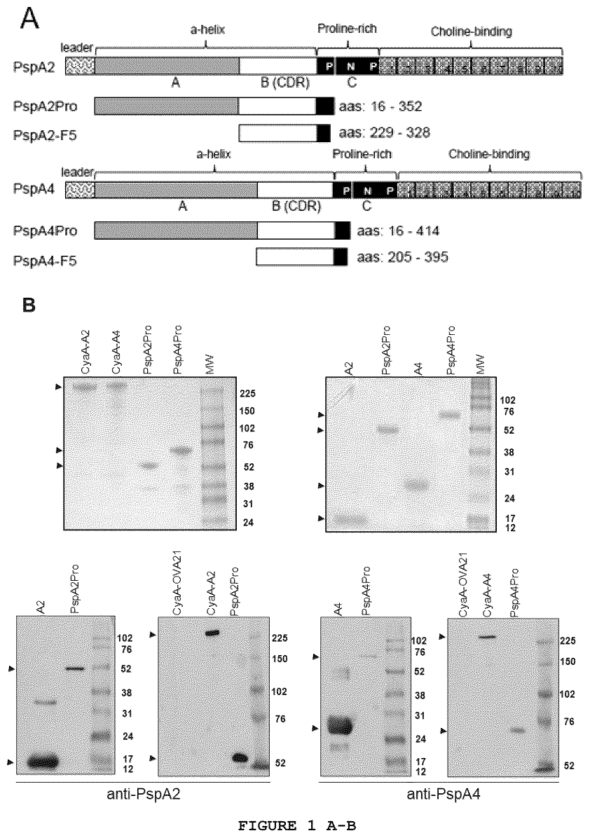

The PspA2-F5 (SEQ ID NO: 1) comprises the final part of the PspA2 (RX1 strain) N-terminal region, including the clade defining region and part of the prolinerich domain (FIG. 1A).

The PspA4-F5 fragment (SEQ ID NO: 2) was defined by the alignment of the amino acid sequences of PspA2-F5 and the PspA4Pro antigen (FIG. 1A). The two fragments were expressed in the permissive domain (residues 224-225) of the CyaA protein, producing the CyaA-PspA2-F5 (SEQ ID NO: 5) and CyaA-PspA4-F5 (SEQ ID NO: 6) with predicted mass weight of 189 kDa and 195 kDa, respectively (FIG. 1B). The PspA2-F5 and PspA4-F5 proteins, with 11.3 kDa and 17.4 kDa, respectively (FIG. 1B), were used as comparison groups in the immunization experiments. All purified proteins were recognized by polyclonal anti-PspA2 or anti-PspA4 antisera (FIG. 1C). PspA2Pro and PspA4Pro, which comprise the entire N-terminal region of the respective PspAs, including part of the proline-rich region, were used as reference and positive controls in SDS-PAGE and Western-blots. (FIGS. 1B and 1C).

Immunization of Mice with CyaA-PspA-F5 Proteins Induce High Levels of Anti-PspA Antibodies:

Mice were immunized with each CyaA-PspA-F5 protein alone or in combination. For the evaluation of the CyaA adjuvant effect, two mice groups were immunized with each PspA-F5 protein alone or in combination. Mice inoculated with saline or CyaA-OVA21 were used as controls.

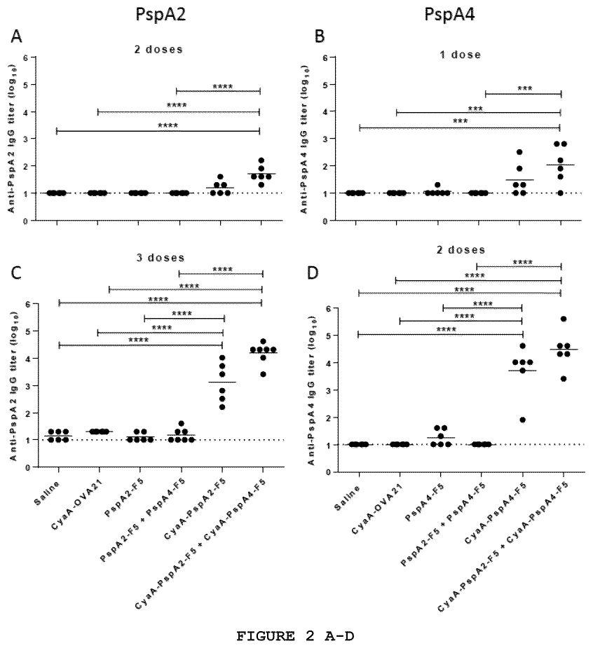

After one dose, no significant increase in the levels of anti-PspA2 IgG were observed in any group. The levels of anti-PspA2 IgG started to increase after the second dose in mice immunized with CyaA-PspA2-F5 (SEQ ID NO: 5) or with this protein combined with CyaA-PspA4-F5 (SEQ ID NO: 6) (FIG. 2A).

Three doses of these two formulations induced high levels of anti-PspA2 IgG in mice, whereas the PspA2-F5 and PspA2-F5+PspA4-F5 vaccines had no significant effect (FIG. 2C). On the other hand, a single dose of CyaA-PspA4 F5 or its combination with CyaA-PspA2-F5 was sufficient to induce anti-PspA4 IgG (FIG. 2B) that further increased after the second dose (FIG. 2D).

Considering the high levels of anti-PspA4 IgG, these mice were not inoculated with a third dose before the challenge with the 3JYP2670 pneumococcal strain, which expresses PspA4 (described below).

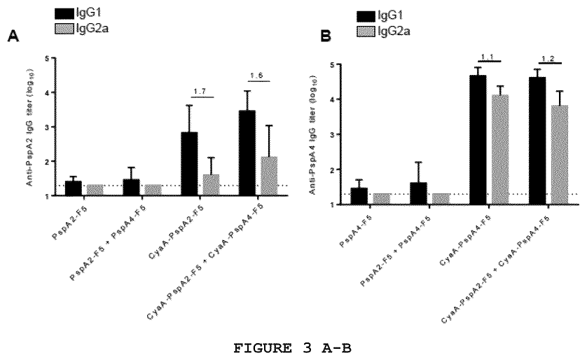

The subtypes of anti-PspA IgG were evaluated by ELISA in sera from mice immunized with three doses of each protein (FIG. 3). In all cases, the levels of IgG1 were higher than levels of IgG2a, although both subtypes were present. The low IgG1/IgG2a ratios observed suggest a balanced Th1/Th2 immune response.

Antibodies Induced by Immunization with CyaA-PspA2 F5 and CyaA-PspA4-F5 Proteins Recognize PspA Expressed by Different Pneumococcal Strains:

To evaluate the capacity of the antibodies induced by immunization with CyaA-PspA2-F5 (SEQ ID NO: 5) and CyaA-PspA4-F5 (SEQ ID NO: 6) to recognize native PspA expressed by pneumococci, it was performed an in vitro binding assay. For these experiments, pooled sera from each mice group that were immunized with the three doses regimen were used.

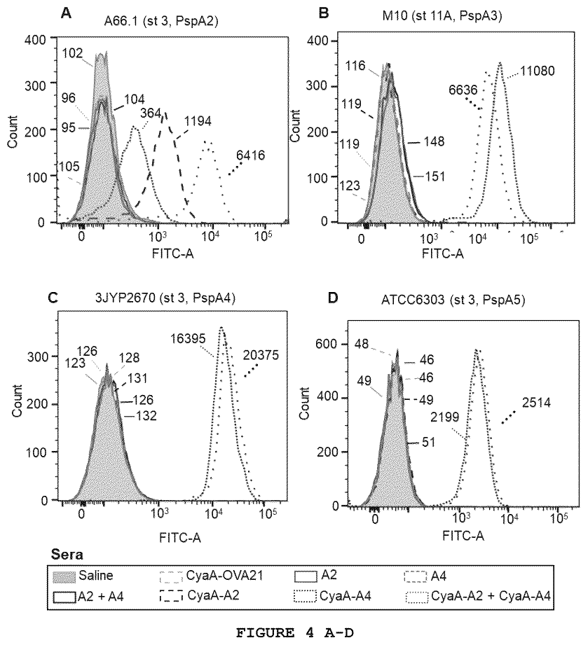

Samples were further incubated with FITC-conjugated anti-mouse IgG and analyzed by flow cytometry. The median of fluorescence intensity of the bacterial populations was compared. Sera from mice immunized with CyaAPspA2-F5 (SEQ ID NO: 5) were able to bind specifically to the strain expressing PspA2 (FIG. 4A), whereas sera from mice immunized with CyaA-PspA4-F5 (SEQ ID NO: 6) reacted with strains expressing PspA3, PspA4 and PspA5 (FIGS. 4B, C and D).

In addition, the CyaA-PspA4-F5 (SEQ ID NO: 6) sera also showed some reactivity with the strain expressing PspA2 (FIG. 4A). Notably, sera from mice immunized with the combined CyaA-PspA2-F5 (SEQ ID NO: 5)+CyaA-PspA4-F5 (SEQ ID NO: 6) formulation were able to bind to the surface of strains expressing PspA2 (A66.1), PspA3 (M10), PspA4 (3JYP2670) and PspA5 (ATCC6303) (FIG. 4).

The capacity of the sera to induce complement deposition on the surface of the bacteria were also evaluated by flow cytometry with comparison of the median of fluorescence intensity of anti-C3-FITC antisera.

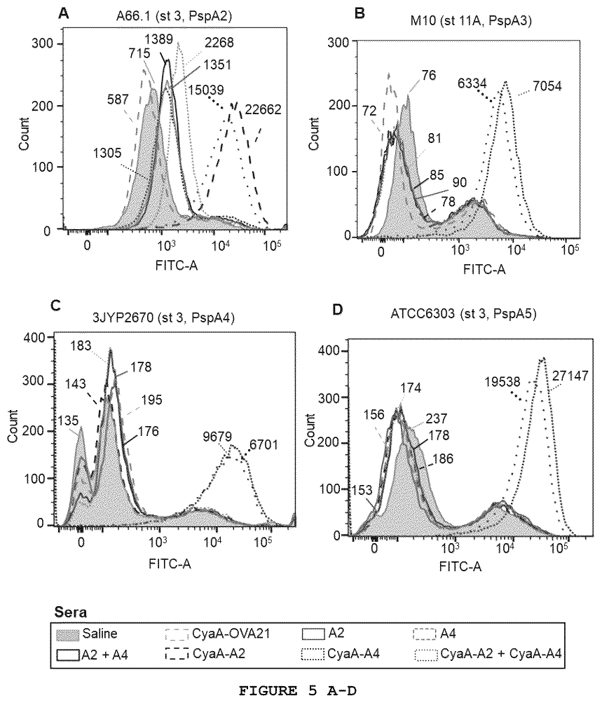

Sera from mice immunized with the combined CyaA-PspA2-F5 (SEQ ID NO: 5)+CyaA-PspA4-F5 (SEQ ID NO: 6) formulation induced complement deposition on the surface of strains expressing PspA2 (FIG. 5A), PspA4 (FIG. 5C) and PspA5 (FIG. 5D). Complement deposition was also observed for the strain expressing PspA3 (FIG. 5B).

For the single-protein formulations, complement deposition was observed for the strain expressing PspA2 incubated with anti-CyaA-PspA2-F5 sera (FIG. 5A) and the strains expressing PspA4 or PspA5 incubated with antiCyaA-PspA4-F5 sera (FIGS. 5C and D, respectively).

Combination of CyaA-PspA2-F5 with CyaA-PspA4-F5 Protects Mice Against Challenge with Strains Expressing Homologous and Heterologous PspAs:

The protective effects of immunization with the CyaA-PspA proteins were analyzed by an invasive respiratory challenge model with serotype 3 pneumococcal strains expressing different PspAs.

Since high levels of anti-PspA4 antibodies were observed after two doses of CyaA-PspA4-F5 (SEQ ID NO: 6) protein (FIG. 2D), mice were challenged with the 3JYP2670 strain (which expresses PspA4) twenty-one days after two immunization doses.

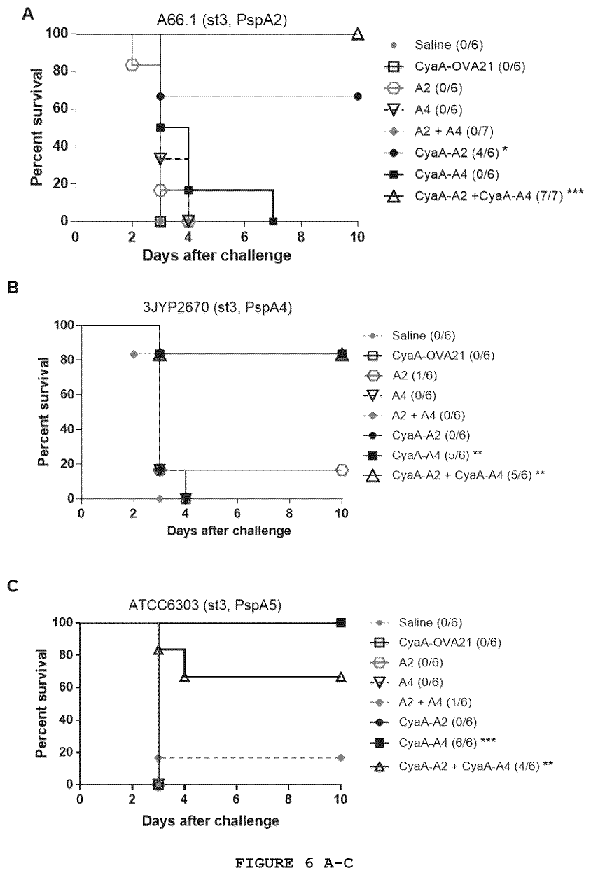

For the analysis of protection against the A66.1 strain (which expresses PspA2) or the ATCC6303 strain (which expresses PspA5) challenges were performed after three doses. In all cases, significant protection, with survival ranging from 66% to 100% depending on the condition tested, were observed (FIG. 6).

Immunization with CyaA-PspA2-F5 (SEQ ID NO: 5) protected mice from the challenge with the PspA2 expressing strain, A66.1 (FIG. 6A), whereas CyaA-PspA4-F5 (SEQ ID NO: 6) strain protected mice from strains expressing PspA4, 3JYP2670 or PspA5, ATCC6303 (FIGS. 6B and C).

The formulation with the combined proteins, CyaA-PspA2-F5 (SEQ ID NO: 5)+CyaA-PspA4-F5 (SEQ ID NO: 6), protected mice from the challenge with the three strains (FIG. 6).

Conversely, for all the challenges, no significant protection was observed in mice immunized with PspA2-F5 (SEQ ID NO: 1) or PspA4-F5 (SEQ ID NO: 2), as well as with CyaA-OVA21, used as a negative control (FIG. 6).

The CyaA-PspA2-F5-PspA4-F5 Fusion Protein Induces Antibodies with Broad Reactivity and Protects Mice Against the Pneumococcal Challenge:

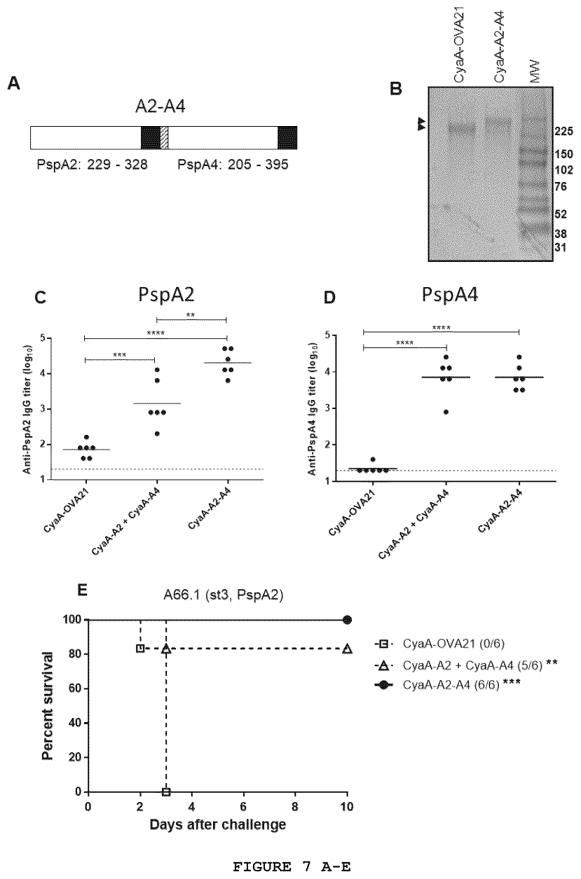

In order to analyze whether a single protein, composed by the F5 fragments from PspA2 and PspA4, would produce a similar effect as the combination of both proteins, a CyaA-PspA2-F5-PspA4-F5 protein (SEQ ID NO: 7) (FIG. 7A) was produced and purified.

Mice were immunized with three doses CyaA-PspA2-F5-PspA4-F5 (SEQ ID NO: 7) or a mixture of CyaA-PspA2 F5 and CyaA-PspA4-F5 as control. The CyaA-PspA2-F5-PspA4-F5 (SEQ ID NO: 7) fusion protein induced higher levels of antiPspA2 IgG when compared with mice immunized with the combined CyaA-PspA2-F5 (SEQ ID NO: 5)+CyaA-PspA4-F5 (SEQ ID NO: 6) (FIG. 7B).

The induction of anti-PspA4 IgG was similar in both mice groups. In addition, the CyaA-PspA2-F5-PspA4 F5 (SEQ ID NO: 7) protein was highly protective against the challenge with the A66.1 strain (which expresses PspA2).

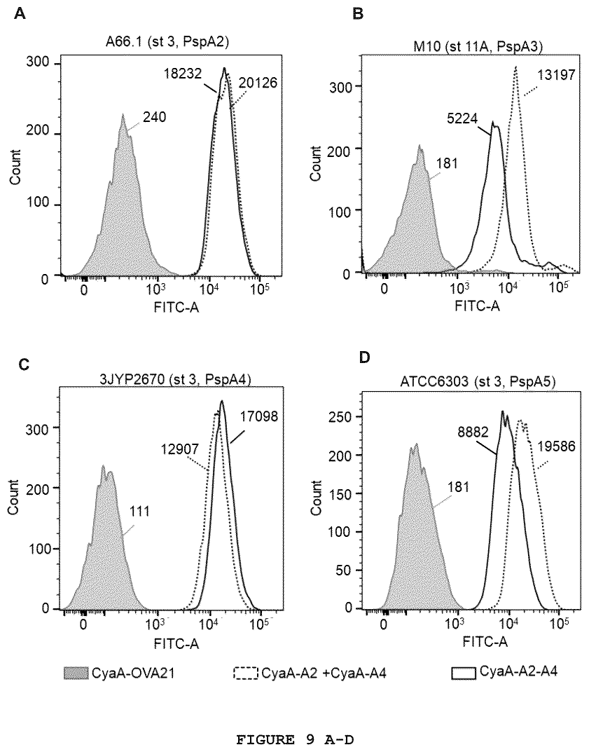

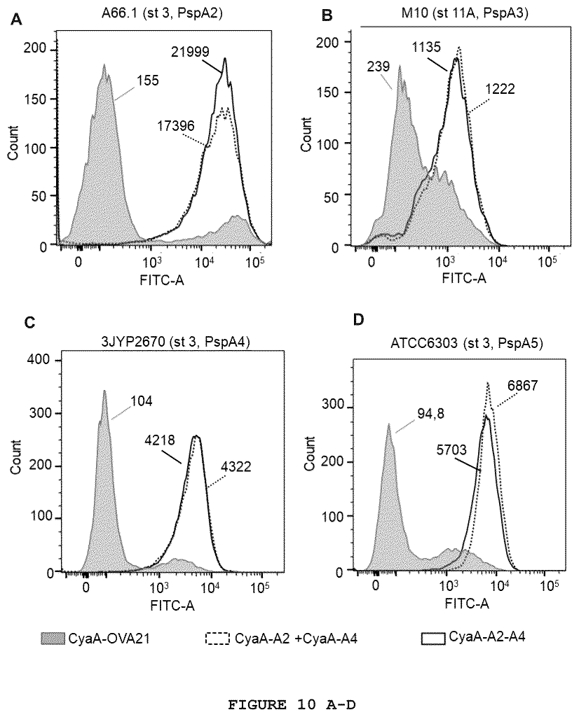

Importantly, antibodies induced by immunization with CyaA-PspA2-F5-PspA4-F5 (SEQ ID NO: 7) showed broad-reactivity (FIG. 9) and induced complement deposition on the surface of pneumococcal strains expressing different PspAs, at levels comparable to antibodies induced by the combination CyaA-PspA2-F5 (SEQ ID NO: 5)+CyaA-PspA4F5 (SEQ ID NO: 6) (FIG. 10).

Furthermore, it is important to emphasize that immune responses were induced without the use of adjuvants, confirming the immunomodulatory properties of CyaA.

Therefore, based on the results obtained, it was observed that the combination of CyaA-PspA2-F5 (SEQ ID NO: 5) and CyaA-PspA4-F5 (SEQ ID NO: 6) conferred high percent survival to mice challenged with strains expressing PspA2, PspA4 and PspA5, indicating the good potential of this formulation regarding coverage of PspA variants. The fusion CyaA-PspA2-F5-PspA4-F5 (SEQ ID NO: 7) showed similar properties regarding the levels and the reactivity of the antibodies against different strains, as well as the ability to induce complement deposition (FIGS. 9 and 10).

The advantage of the fusion protein would be the purification of a single recombinant protein, simplifying the process and the product. In one embodiment of the present invention, analysis of the efficacy of CyaAPspA2-F5 (SEQ ID NO: 5) and CyaAPspA4-F5 (SEQ ID NO: 6) proteins against challenge models of pneumococcal systemic infection have been shown.

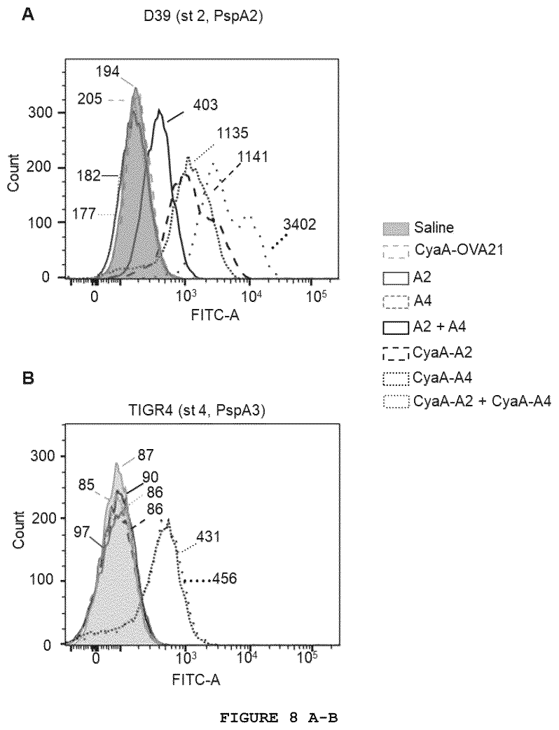

Furthermore, reactivity of the antibodies in vitro was shown to strains expressing other serotypes (M10 strain, st11A, PspA3; D39 strain, st2, PspA2 and TIGR4 strain, st4, PspA3) (FIG. 4 and FIG. 8).

Antibodies elicited by the immunization with CyaA-PspA2-F5 (SEQ ID NO: 5) and CyaA-PspA4-F5 (SEQ ID NO: 6) proteins also induced the deposition of complement on pneumococcal surface of different strains and protection against the invasive challenges correlated with this capacity.

In conclusion, the present invention proposes recombinant proteins comprising fragments of PspA and CyaA to induce high levels of anti-PspA antibodies. In one embodiment of the present invention, it was shown that a mixture of CyaA-PspA2-F5 (SEQ ID NO: 5) and CyaA-PspA4-F5 (SEQ ID NO: 6) proteins or the fusion CyaA-PspA2-F5-PspA4 F5 (SEQ ID NO: 7) conferred protection to mice against systemic infection with pneumococci expressing homologous or heterologous PspAs. Furthermore, in vitro results also indicated that these proteins have good potential to provide broad protection against different isolates.

### Experimental Results of PspA1 and PspA3

In this invention the adenylate cyclase toxin system from Bordetella pertussis (CyaA) for the expression and presentation of Pneumococcal Surface protein A (PspA) fragments has been tested with the aim to propose a universal vaccine against Streptococcus pneumoniae (pneumococci).

Previous data, as mentioned above, have shown that CyaA expressing fragments from PspAs from family 1 and 2, particularly the fragments 5 of PspA2 (PspA2-F5, also named A2) and PspA4 (PspA4-F5, also named A4) induce high levels of antibodies in mice. Sera from mice immunized with the combination CyaA-A2+CyaA-A4 or the fusion protein CyaAA2-A4, were able to bind to the surface of pneumococcal isolates form different serotypes expressing pspA2, PspA3, PspA4 or PspA5. The vaccine candidates also protected mice against invasive challenges (sepsis model) with pneumococcal isolates expressing PspA2, PspA4 and PspA5.

Now, the capacity of the CyaA-A2-A4 protein to protect mice against pulmonary colonization (pneumonia model) and to induce immune responses in the lungs was analyzed. The experiment was performed in comparison with the PCV13 (Prevnar, Pfizer), a pneumococcal conjugate vaccine current in the market. The importance of this analysis relies in the fact that pneumococcus is a respiratory pathogen and pneumonia is a major disease caused by the bacteria, that may cause death as such, or may evolve to sepsis or meningitis. In addition, the protection against a ST3 pneumococcal isolate was tested. Data in the literature show that ST3 may escape immunity induced by the conjugated vaccines that have been used in populations in the last years.

In addition, it was observed that sera from mice immunized with the formulations containing PspA2-F5 and PspA4-F5 (CyaA-A2+CyaA-A4 or CyaA-A2-A4) displayed reduced reactivity with pneumococcal isolates expressing PspA1. Therefore, new constructs including the PspA1-F5 fragment (A1) were tested to evaluate a potential increase in reactivity and protection against pneumococcal isolates expressing PspA1.

With this rationale, although it was observed reactivity of the sera induced by CyaA-A2+CyaA-A4 or CyaA-A2-A4 immunization with pneumococcal isolates expressing PspA3, proteins containing the PspA3-F5 fragment (A3) were also tested.

### Methods—Ethical Statement:

This study was performed according to the guidelines outlined by the Brazilian National Council for Control of Animal Experimentation (CONCEA), which follows international guidelines for animal welfare and the principles of the 3Rs. Experimental protocols comply with the ARRIVE guidelines and were approved by the Ethic Committee on Animal Use of the Instituto Butantan, São Paulo, Brazil (protocol numbers 8112091117 and 5370090919). Mice were housed in a BSL2 animal facility, in micro isolators with individual ventilation and temperature and light cycle control. Animals received food and water ad libitum and manipulation was performed by trained personnel.

### Bacteria:

S. pneumoniae strains ATCC6303 (st 3, PspA5); 122/00 (st23F, PspA5); 3JYP2670 (st 3, PspA4); 255/00 (ST14, PspA4); A66.1 (st 3, PspA2); ATCC6301 (st1, PspA1); EF3030 (st 19F, PspA1); 0603 (st 6B, PspA1); D39 (st2, PspA2); M10 (st 11A, PspA3); TIGR4 (st 4, PspA3) were grown on Triptic soy agar containing 5% defibrinated sheep blood (blood agar, Laborclin, Brazil) at 37° C. Stocks for animal challenge were prepared in liquid Todd-Hewitt media (Difco) supplemented with 0.5% yeast extract (THY). Bacteria were grown until exponential phase (OD600 nm=0.4), centrifuged, and suspended in 1/10 of the initial volume in THY containing 20% glycerol. Stocks were maintained at −80° C. and quantified by plating on blood agar.

### Immunization of Mice and Analysis of Immune Responses:

Female SPF BALB/c mice were produced by the animal facility from the University of São Paulo Medical School (Brazil). Protein doses were adjusted for 50 μmol per mice, considering the differences in molecular weight between the CyaA-PspA proteins (around 200 kDa, 10 μg per dose). For the analysis of the mixture of CyaA-A2+CyaA-A4 (10 μg of each protein), in all experiments, control groups received purified CyaA-OVA21 (10 μg per dose) or saline. Residual LPS was quantified in all protein preparations using the Quantitative Chromogenic LAL assay (QCL-1000, Lonza, USA), according to the manufacturer instructions. The LPS levels in the doses administered to mice were below 1 EU/mL. Mice (6 per group) received two or three subcutaneous doses in 15-days intervals. Blood was collected 14 days after each immunization and induction of anti-PspA IgG was evaluated by ELISA using plates coated with 1 μg/mL of PspA1, PspA2Pro, PspA3, PspA4Pro or PspA5 and serial dilutions of the samples (starting at 1:20 for sera or 1:4 for Bronchioalveolar lavage fluids, BALFs). Horseradish Peroxidase (HRP) conjugated anti-mouse IgG (Sigma-Aldrich, USA) was used as secondary antibody for sera samples and Alkaline Phosphatase conjugated anti-mouse IgG was used as secondary antibody for BALF samples (Sigma). Reactions were developed using o-Phenylenediamine dihydrochloride (sera) or 4-Nitrophenyl Phosphate (Balf) as substrates and the absorbances were measured at 492 nm or 405 nm, respectively in a spectrophotometer multiskan EX (Thermo Fisher Scientific, USA). Antibody titers were defined as the reciprocal of the dilution that produced an absorbance of 0.1. Sera were also tested for binding to the surface of different pneumococcal strains as previously described. Briefly, bacterial suspensions (10⁸ CFU/mL) were incubated with 5% (V/V) of heat-inactivated sera pooled from each experimental group, for 30 min on ice. Samples were washed once with PBS and incubated for with fluorescein isothiocyanate (FITC)-conjugated goat anti-mouse IgG (MP Biomedicals, USA), for 30 min on ice. Samples were suspended in cytofix (BD Biosciences, USA) and analyzed by flow cytometry in a FACS Canto II equipment (BD Biosciences, USA), with 15,000 gated events recorded. Medians of fluorescence intensity were compared using the Flow Jo 10.1 software.

### Pneumococcal Challenges:

Twenty-one days after the last immunization, animals were anesthetized through the intraperitoneal route (i.p.) with 100 mg/Kg of ketamine chloride (Ceva, Brazil) and 20 mg/Kg of xylazine chloride (Ceva). S. ilneumoniae 3JYP2670 (3×10⁵ CFU per mice) or EF3030 (1×10⁶ CFU per mice) were inoculated through the intranasal route, in 50 μL of saline. Animals were monitored twice a day, for 10 days, and those that presented signs of disease with piloerection, hunched posture and reduction in activity, were euthanized immediately with a lethal dose of anesthetics (60 mg/Kg of xylazine chloride and 300 mg/Kg of ketamine chloride, i.p.). Mice that remained until the end of the experiment were healthy and active; no animals died before meeting the criteria for euthanasia. For the pneumonia model, after the euthanasia, Bronchioalveolar lavage fluids (BALFs) or the lungs were collected as previously described. Samples were plated on blood agar for CFU counting. BALFs supernatants were aliquoted and used for the detection of antibodies by ELISA.

### Statistical Analysis:

Antibody titers among all groups were compared by One-way ANOVA, and the comparisons between two groups were performed by Tukey's post-test. Animal Survival was analyzed by the Log-rank survival curve using the Mantel-Cox test for the comparison between groups. Prism GraphPad 6.0 software was used for the analyses and P<0.05 was considered significantly different.

### Results:

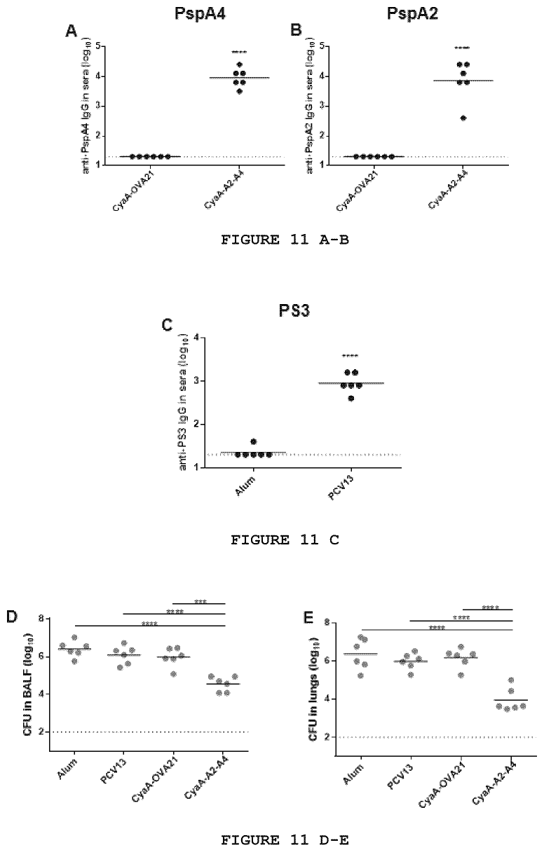

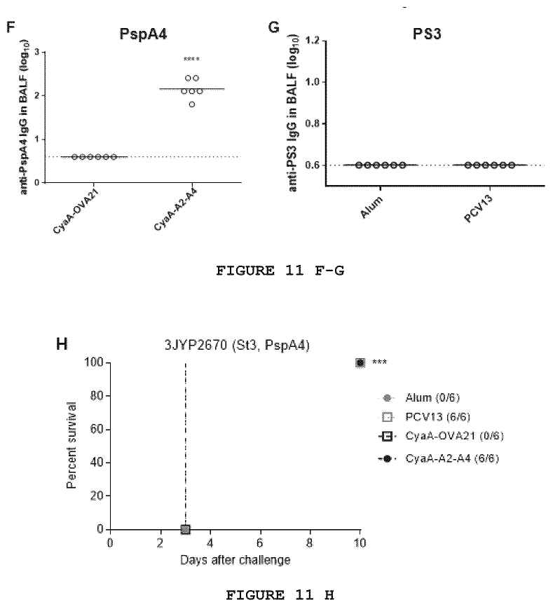

In order to evaluate the protective activity of the CyaA-PspA proteins against pneumococcal colonization in the lungs (pneumonia model), animals were immunized with 3 doses of CyaA-A2-A4 or the control CyaA-OVA21. In this experiment, the pneumococcal conjugate vaccine PCV13 with the respective control Alum were also tested. Induction of high levels of anti-PspA4 and anti-PspA2 IgG was observed in mice immunized with CyaA-A2-A4 (FIGS. 11A and B). On the other hand, immunization with PCV13 induced high levels of IgG anti PS3 (purified polysaccharide from a serotype 3 (ST3) pneumococcal isolate) (FIG. 11C). It is important to mention that it was chosen to analyze antibodies against PS3, since the experiment was programed to evaluate protection against a ST3 pneumococcal isolate. Mice were then infected with the 3JYP2670 pneumococcal strain (ST3, PspA4) through the nasal route and BALFs and lungs were collected 12 h after the challenge to evaluate pulmonary colonization (FIGS. 11D and 11E). Immunization with CyaAA2-A4, promoted a significant reduction of pneumococcal colonization in mice respiratory tract 12 h after the challenge, reflected by the low numbers of bacteria recovered from BALF (FIG. 11D) and lungs (FIG. 11E). Interestingly, reduction was not observed in mice immunized with the PCV13 vaccine (FIGS. 11D and 11E). The levels of specific antibodies in BALFs, after the challenge, were also evaluated. High levels of anti-PspA4 IgG were observed in the respiratory tract of mice immunized with CyaA-A2-A4 after the challenge (FIG. 11F). Since immunization was through the subcutaneous route, it is possible that antibodies from the bloodstream have passed to the airways before the challenge or an influx of antibodies may have been induced by the challenge. On the other hand, no anti-PS3 antibodies were observed in the airways of mice immunized with PCV13 (FIG. 11G). Thus, a marked difference can be observed between the two vaccines. The reduction of lung colonization, at least at the time point tested, was more effective for the CyaA-A2-A4 vaccine when compared with PCV13 and antibodies may have an important role on this effect. However, a duplicate of this experiment was performed to follow survival along 10 days, and both vaccines CyaA-A2-A4 and PCV13 were able to confer protection to 100% of mice (FIG. 11H). In summary, our results indicate that the CyaAA2-A4 vaccine is more effective against pulmonary infection with ST3 pneumococci when compared to PCV13, but both vaccines are highly effective against systemic infection (sepsis model).

Although the combination of PspA2 and PspA4 in the vaccine was proposed by the present invention to provide cross-reactivity against pneumococcal isolates expressing PspAs from family 1 (PspA1 and PspA2) and family 2 (PspA3, PspA4 and PspA5), comprising more than 99% of the pneumococcal isolates causing disease, reactivity against isolates expressing PspA1 was surprisingly low.

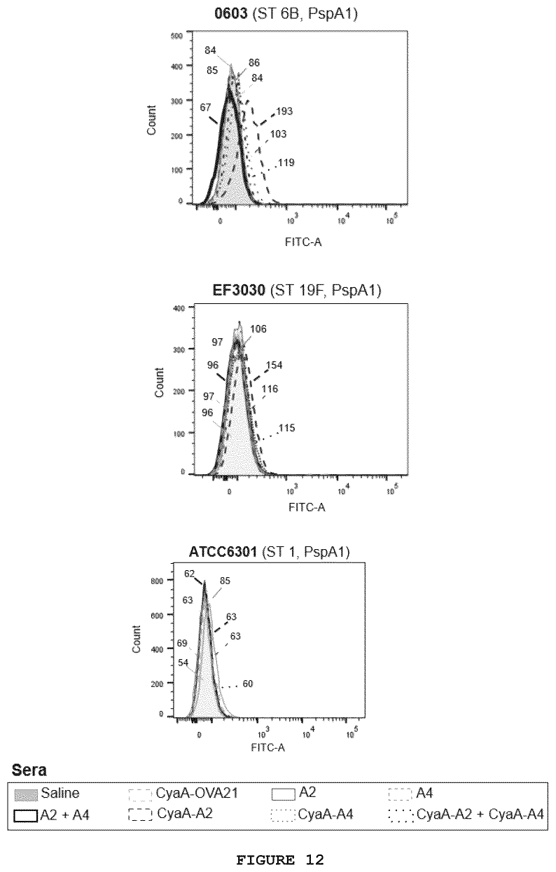

As can be observed in FIG. 12, sera from mice immunized with CyaA-A2 alone or combined with CyaA-A4, showed no or very low reactivity with three strains expressing PspA1 (FIG. 12). Therefore, CyaA proteins containing the PspA1-F5 (A1), seq. ID NO: 3, were constructed for testing. In addition, the present inventors also decided to evaluate proteins containing the PspA3-F5 fragment (A3), seq. ID NO: 4, although the CyaA-A2-A4 protein had already shown some reactivity with pneumococci expressing PspA3.

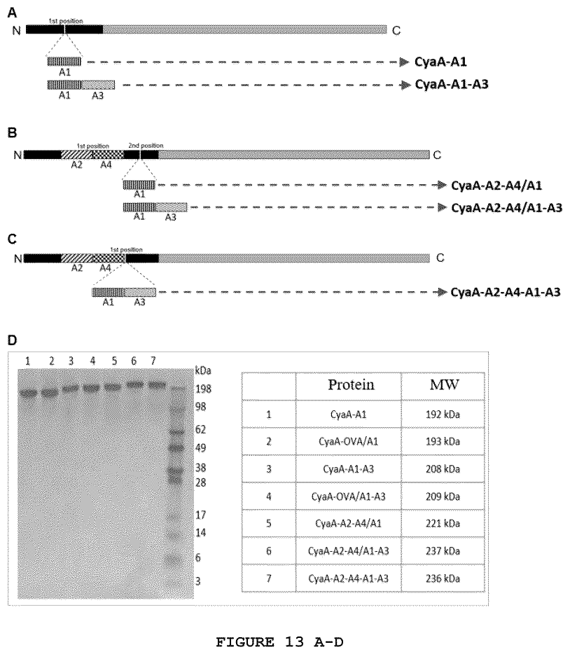

In FIG. 13, a schematic representation of the constructed proteins containing the A1, the A3 or both A1 and A3 fragments is shown. CyaA-A1 (SEQ. ID NO: 8) or CyaA-A1-A3 (SEQ. ID NO: 9), with insertions of the fragments at position 224-225 of the CyaA protein were constructed to allow testing of combinations with CyaA-A2-A4 (SEQ. ID NO: 7). Additionally, the A1 or A1-A3 fragments were inserted in the position 319-320 position, producing the CyaA-A2-A4/A1 protein (SEQ. ID NO: 10) and CyaA-A2-A4/A1-A3 (SEQ. ID NO: 11). Finally, a fusion protein containing the A2-A4-A1-A3 fragments at position 224-225 was constructed.

The proteins alone or in combinations with CyaA-A2-A4 were tested initially in a screening experiment, to evaluate induction of antibodies against PspA1 and PspA3 as well as protection against pulmonary infection with a pneumococcal isolate expressing PspA1. From this screening, the formulations CyaA-A2-A4+CyaA-A1, CyaA-A2-A4/A1 and CyaA-A2-A4/A1-A3 produced the best results and were selected for further evaluation.

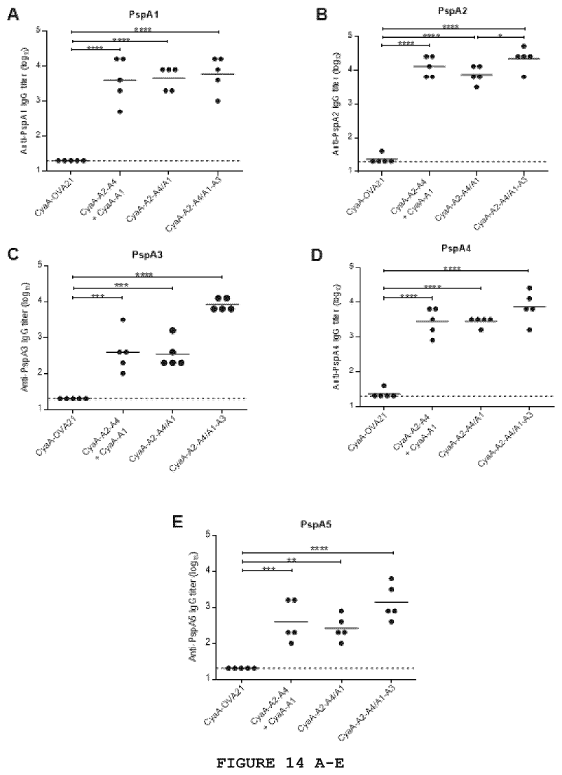

Mice were immunized with three doses of CyaA-A2-A4+CyaA-A1, CyaA-A2-A4/A1 or CyaA-A2-A4/A1-A3 and the induction of antibodies against PspA1, PspA2, PspA3, PspA4 and PspA5 was evaluated by ELISA (FIG. 14). The three formulations induced high levels of anti-PspA1 (FIG. 14A) anti-PspA2 (FIG. 14B) and anti-PspA4 (FIG. 14D). Although the CyaA-A2-A4+CyaA-A1 and the CyaA-A2-A4/A1 induced significant levels of IgG reacting with PspA3, the highest levels were observed in sera from mice immunized with CyaA-A2-A4/A1-A3 (FIG. 14C). For PspA5, all the formulations induced similar levels of IgG reacting with the protein, the levels induced by the CyaA-A2-A4/A1-A3 protein were slightly higher (FIG. 14E).

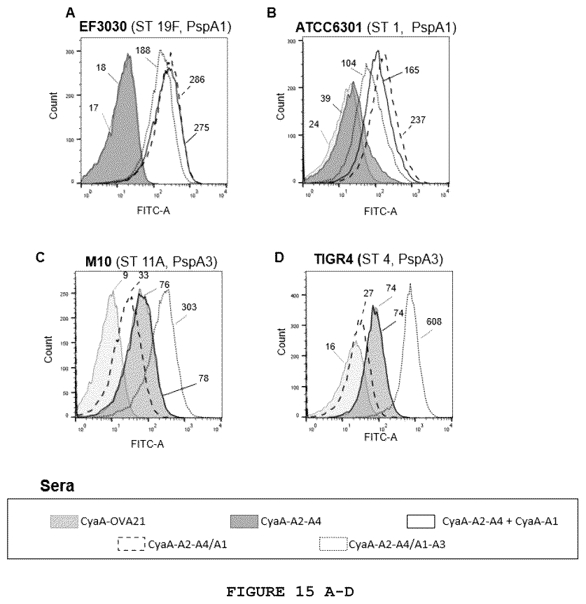

Then, the present inventors evaluated the capacity of the sera to recognize the different PspA at their native conformation, at the surface of the bacteria, through flow cytometry assays, using FITC conjugated secondary antibodies. The mean fluorescence intensity (MFI) of the curves was compared (FIG. 15).

The presence of the A1 fragment in the formulations improved reactivity of the sera with pneumococcal isolates expressing PspA1, when compared with sera from mice immunized with CyaA-A2-A4 (FIGS. 15A and B). The MFIs of the curves produced by the sera from mice immunized with CyaA-A2-A4 were low and very similar to the observed for the sera of the negative control (CyaA-OVA21), for both EF3030 and ATCC6301 pneumococcal isolates. On the other hand, high MFI levels were observed for the sera from mice immunized with CyaA-A2-A4+CyaA-A1, CyaA-A2-A4/A1 or CyaA-A2-A4/A1-A3 (FIGS. 15A and B), with little differences among the curves.

The same sera were tested against the pneumococcal isolates M10 and TIGR4, which express PspA3. IgG in the sera of mice immunized with CyaA-A2-A4+CyaA-A1, CyaA-A2-A4/A1 or CyaA-A2-A4/A1-A3 were able to bind to the surface of both bacteria, producing higher MFI than the negative control CyaA-OVA21. However, sera from mice immunized with the CyaA-A2-A4/A1-A3 protein was superior than the others, indicating a better capacity of this sera to recognize native PspA3 (FIGS. 15C and D).

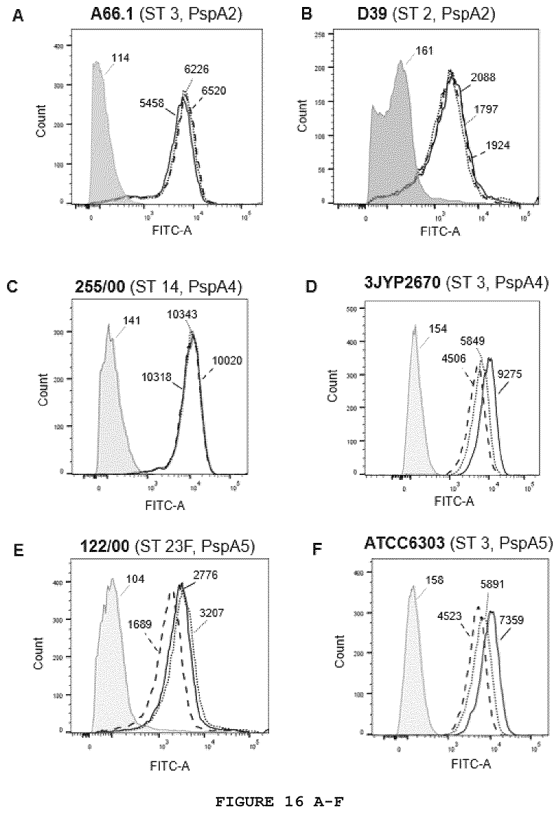

To evaluate if the sera from the selected formulations maintained the high capacity to bind to pneumococcal isolates expressing PspA2, PspA4 or PspA5, flow cytometry experiments were performed with two strains bearing each of these PspAs clades (FIG. 16). Overall, sera from mice immunized with the three formulations, CyaAA2-A4+CyaA-A1, CyaA-A2-A4/A1 or CyaA-A2-A4/A1-A3, showed high capacity to bind to all the pneumococcal isolates tested, either expressing PspA2 (FIGS. 16A and B), PspA4 (FIGS. 16C and D) or PspA5 (FIGS. 16E and F). No significant differences were observed among the sera.

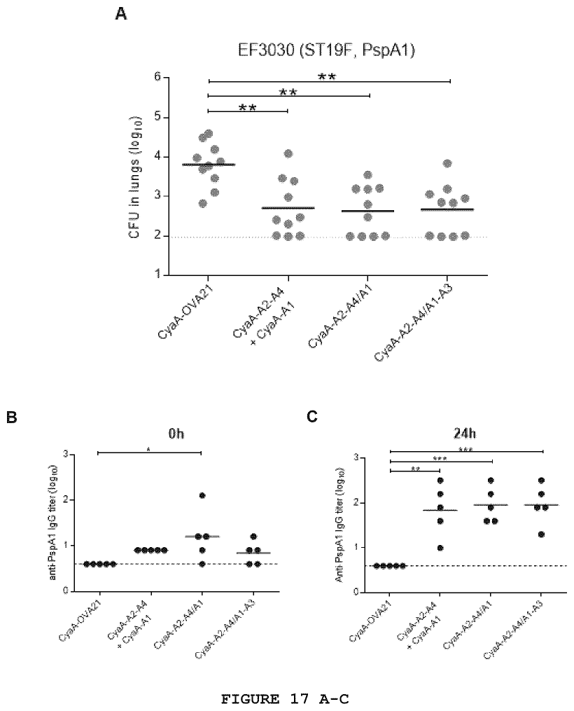

Since the proteins containing the A1 fragment improved reactivity against pneumococcal isolates expressing PspA1, a respiratory challenge was performed in mice immunized with CyaA-A2-A4+CyaA-A1, CyaA-A2-A4/A1 or CyaAA2-A4/A1-A3. A control group was immunized with the CyaAOVA21 protein. The EF3030 pneumococcal isolate (ST 19F, PspA1) was inoculated in immunized mice, through the nasal route, and 24 h later the lungs were collected to evaluate pulmonary colonization. All three formulations were able to significantly reduce the pneumococcal numbers in mice lungs, when compared to the control group (FIG. 17A). The present inventors also evaluated the levels of anti-PspA1 antibodies in the respiratory tract of mice before (0 h, FIG. 17B) and 24 h after the challenge (FIG. 17C). Surprisingly, antiPspA1 IgGs, at low levels, were observed in BALFs from mice before the challenge, indicating that the subcutaneous immunization with the CyaA-PspA proteins can induce some level of antibodies in the respiratory mucosa (FIG. 17B). These levels further increased after the challenge (FIG. 17C).

### Conclusions:

Together with the previous data, the CyaA-PspA proteins, containing the A2 and A4 fragments induce antibodies that recognize PspA2, PspA3, PspA4 and PspA5. These proteins also confer protection to mice against pneumococcal invasive challenges expressing PspA2, PspA4 and PspA5 (previous results). Here it was showed that the CyaA2 A4 protein is also able to protect mice against lung colonization (pneumonia model) with a pneumococcal isolate expressing PspA4. However, CyaA-A2-A4 did not induce antibodies that recognize PspA1. The inclusion of the A1 fragment in the formulation improved the reactivity against pneumococcal strains expressing PspA1 and conferred protection against lung colonization with a pneumococcal strain expressing PspA1. Inclusion of the A3 fragment also improved reactivity against isolates expressing PspA3. Immunization with the CyaA-PspA through the subcutaneous route can induce some level of antibodies in the respiratory mucosa that are further increased by the challenge. AntiPspA antibodies in the lungs, induced by vaccination with CyaA-PspA proteins may be important for protection against lung colonization.

Therefore, several CyaA-PspA proteins that allow different combinations of antigens and provide broad protection against pneumococcal infections were constructed. The formulations described here may be proposed as a universal vaccine against invasive pneumococcal diseases as well as pneumonia caused by different pneumococcal serotypes.

In summary, the invention refers to the following aspects, as defined in the following numbered items:

1. A recombinant protein comprising one or more fragments of pneumococcal surface protein A (PspA) and the modified adenylate cyclase (CyaA) from Bordetella species, especially Bordetella pertussis.

2. Recombinant protein, according to aspect 1, wherein said Bordetella species is Bordetella pertussis.

3. Recombinant protein, according to aspect 1 or 2, wherein the enzymatic activity of said CyaA is inactivated.

4. Recombinant protein, according to any one of aspects 1 to 3, wherein said PspA protein fragment is selected from families 1 and 2, preferably fragments of clades 1 to 4, or a combination of two or more thereof.

5. Recombinant protein according to any one of aspects 1 to 4, wherein said fragment is selected from the group consisting of:
fragments encoding a final part of the N-terminal region of PspAs of clades 2 (PspA2-F5) and 4 (PspA4-F5), wherein PspA2-F5 consists of the amino acid sequence as set forth in SEQ ID NO: 1, and PspA4-F5 consists of the amino acid sequence as set forth in SEQ ID NO: 2;
fragments encoding a final part of the N-terminal region of PspAs of clades 1 (PspA1-F5) and 3 (PspA3-F5), wherein PspA1-F5 consists of the amino acid sequence as set forth in SEQ ID NO: 3, and PspA3-F5 consists of the amino acid sequence as set forth in SEQ ID NO: 4;
fragments that encode a final part of the N-terminal region of PspAs from clades 2 (PspA2-F5), 4 (PspA4-F5) and 1 (PspA1-F5); and
fragments that encode a final part of the N-terminal region of PspAs from clades 2 (PspA2-F5), 4 (PspA4-F5), 1 (PspA1-F5) and 3 (PspA3-F5).

6. Recombinant protein, according to any one of aspects 1 to 5, wherein said PspAs fragments are encoded by the following nucleotides sequences:
The DNA sequence as set forth in SEQ ID NO: 29 and degenerate sequences thereof that encode the PspA2-F5 as set forth in SEQ ID NO: 1;
The DNA sequence as set forth in SEQ ID NO: 30 and degenerate sequences thereof that encode the PspA4-F5 as set forth in SEQ ID NO: 2;
The DNA sequence as set forth in SEQ ID NO: 31 and degenerate sequences thereof that encode the PspA1-F5 as set forth in SEQ ID NO: 3; and/or
The DNA sequence as set forth in SEQ ID NO: 32 and degenerate sequences thereof that encode the PspA3-F5 as set forth in SEQ ID NO: 4.

7. Recombinant protein according to any one of aspects 1 to 6, comprising or consisting of the following amino acid sequences:
CyaA-PspA2-F5 consisting of the amino acid sequence as set forth in SEQ ID NO: 5; and/or
CyaA-PspA4-F5 consisting of the amino acid sequence as set forth in SEQ ID NO: 6; and/or
CyaA-PspA2-F5-PspA4-F5 consisting of the amino acid sequence as set forth in SEQ ID NO: 7; and/or
CyaA-PspA1-F5 consisting of the amino acid sequence as set forth in SEQ ID NO: 8; and/or
CyaA-PspA1-F5-PspA3-F5 consisting of the amino acid sequence as set forth in SEQ ID NO: 9; and/or
CyaA-PspA2-F5-PspA4-F5-PspA1-F5 consisting of the amino acid sequence as set forth in SEQ ID NO: 10; and/or
CyaA-PspA2-F5-PspA4-F5-PspA1-F5-PspA3-F5 consisting of the amino acid sequence as set forth in SEQ ID NO: 11.

8. Recombinant protein according to any one of aspects 1 to 7, which are encoded by the following nucleotide sequences:
DNA sequence as set forth in SEQ ID NO: 12 and degenerate sequences thereof encoding a recombinant protein CyaA-PspA2-F5 as set forth in SEQ ID NO: 5;
DNA sequence as set forth in SEQ ID NO: 13 and degenerate sequences thereof that encode a recombinant protein CyaA-PspA4-F5 as set forth in SEQ ID NO: 6;
DNA sequence as set forth in SEQ ID NO: 14 and degenerate sequences thereof that encode a recombinant protein CyaA-PspA2-F5-PspA4-F5 as set forth in SEQ ID NO: 7;
DNA sequence as set forth in SEQ ID NO: 15 and degenerate sequences thereof that encode a recombinant protein CyaA-PspA1-F5 as set forth in SEQ ID NO: 8;
DNA sequence as set forth in SEQ ID NO: 16 and degenerate sequences thereof that encode a recombinant protein CyaA-PspA1-F5-PspA3-F5 as set forth in SEQ ID NO: 9;
DNA sequence as set forth in SEQ ID NO: 17 and degenerate sequences thereof that encode a recombinant protein CyaA-PspA2-F5-PspA4-F5-PspA1-F5 as set forth in SEQ ID NO: 10; and/or
DNA sequence as set forth in SEQ ID NO: 18 and degenerate sequences thereof that encode a recombinant protein CyaA-PspA2-F5-PspA4-F5-PspA1-F5-PspA3-F5 as set forth in SEQ ID NO: 11.

9. Expression cassette comprising DNA sequence selected from the group consisting of the nucleotide sequence as set forth in SEQ ID NOs: 12 to 18 and degenerate sequences thereof encoding a recombinant protein as set forth in SEQ ID NOs: 5 to 11, respectively.

10. Immunogenic composition comprising the recombinant protein as defined in any one of aspects 1 to 8, or the expression cassette as defined in item 9, and additionally a pharmaceutically acceptable carrier and/or adjuvant.

11. Immunogenic composition according to aspect 10, wherein it is in the form of a vaccine.

12. Immunogenic composition, according to aspect 10 or 11, wherein it is administered parenterally or is administered via the mucosal route.

13. Immunogenic composition, according to any one of aspects 10 to 12, wherein it is for use in preventing infections caused by Streptococcus pneumoniae.

14. Use of the recombinant protein as defined in any of aspects 1 to 8, the expression cassette as defined in aspect 9, or the immunogenic composition as defined in any of aspects 8 to 11 wherein it is for the manufacture of a vaccine for preventing infections caused by Streptococcus pneumoniae.

15. Use, according to aspect 14, wherein said medicine is a vaccine to prevent infections caused by Streptococcus pneumoniae, which offers broad-spectrum protection against different pneumococcal isolates, regardless of serotypes.

Thus, the embodiments presented in the present disclosure do not limit the totality of possibilities, and it will be understood that various omissions, substitutions and changes can be made by a person skilled in the art, without departing from the scope of the present invention.

It is understood that all combinations of the elements that perform the same function or substantially use the same ways to achieve similar results are within the scope of this invention. Substitutions of elements from one described embodiment to another are also fully intended and contemplated.

A person skilled in the art will value the knowledge presented here and will be able to reproduce the invention in the presented embodiments and in other variants, covered in the scope of the claims.

### BIBLIOGRAPHIC REFERENCES

Vadesilho C F, Ferreira D M, Gordon S B, Briles D E, Moreno A T, et al. (2014) Mapping of epitopes recognized by antibodies induced by immunization of mice with PspA and PspC. Clin Vaccine Immunol 21: 940-948.
Darrieux M, Moreno A T, Ferreira D M, Pimenta F C, de Andrade A L, et al. (2008) Recognition of pneumococcal isolates by antisera raised against PspA fragments from different clades. J Med Microbiol 57: 273-278.
Glaser P, Elmaoglou-Lazaridou A, Krin E, Ladant D, Bârzu O, Danchin A. (1989) Identification of residues essential for catalysis and binding of calmodulin in Bordetella pertussis adenylate cyclase by site-directed mutagenesis. The EMBO Journal 8:967-972
Glaser P, Munier H, Gilles A M, Krin E, Porumb T, Bârzu O, Sarfati R, Pellecuer C, Danchin A. (1991) Functional consequences of single amino acid substitutions in calmodulin-activated adenylate cyclase of Bordetella pertussis. The EMBO Journal 10:1683-1688
Guermonprez P, Fayolle C, Karimova G, Ullmann A, Leclerc C, et al. (2000) Bordetella pertussis adenylate cyclase toxin: a vehicle to deliver CD8-positive Tcell epitopes into antigen-presenting cells. Methods Enzymol 326: 527-542.
Karimova G, Fayolle C, Gmira S, Ullmann A, Leclerc C, et al. (1998) Charge dependent translocation of Bordetella pertussis adenylate cyclase toxin into eukaryotic cells: implication for the in vivo delivery of CD8(+) T cell epitopes into antigen-presenting cells. Proc Natl Acad Sci USA 95: 12532-12537.
Ladant, D, Glaser, P, Ullman, A (1992) Insertional mutagenesis of Bordetella pertussis adenylate cyclase. J. Biol. Chem. 267(4): 2244-2250.
Ramos C R, Abreu P A, Nascimento A L, Ho P L (2004) A high-copy T7 Escherichia coli expression vector for the production of recombinant proteins with a minimal N-terminal His-tagged fusion peptide. Braz J Med Biol Res 37: 11031109.
Preville X., Ladant D., Timmerman B., and C. Leclerc. (2005) Eradication of established tumors by vaccination with recombinant Bordetella pertussis adenylate cyclase carrying the human papillomavirus 16 E7 oncoprotein. Cancer Research 65:641-649
Teodorowicz M, Perdijk O, Verhoek I, Govers C, Savelkoul H F, et al. (2017) Optimized Triton X-114 assisted lipopolysaccharide (LPS) removal method reveals the immunomodulatory effect of food proteins. PLoS One 12: e0173778.
Salcedo-Rivillas C, Debrie A S, Miyaji E N, Ferreira J M, Jr., Raw I, et al. (2014) Pertussis toxin improves immune responses to a combined pneumococcal antigen and leads to enhanced protection against Streptococcus pneumoniae. Clin Vaccine Immunol 21: 972-981.
Agudelo, C. I. et al. The direct effect of pneumococcal conjugate vaccines on invasive pneumococcal disease in children in the Latin American and Caribbean region (SIREVA 2006-17): a multicentre, retrospective observational study. Lancet Infect Dis, v. 21, n. 3, p. 405 417, March 2021.
Sings, H. L. et al. Pneumococcal Conjugate Vaccine Impact on Serotype 3: A Review of Surveillance Data. Infect Dis Ther, v. 10, n. 1, p. 521-539, March 2021.
Oliveira M L, Miyaji E N, Ferreira D M, Moreno A T, Ferreira P C, Lima F A, et al. Combination of pneumococcal surface protein A (PspA) with whole cell pertussis vaccine increases protection against pneumococcal challenge in mice. PloS one. 2010; 5: e10863.

1. Recombinant protein characterized in that it comprises one or more fragments of pneumococcal surface protein A (PspA), a fragment from the N-terminal region of PspA protein from family 1 from Streptococcus pneumoniae, a fragment from the N-terminal region of PspA protein rom family 2 from Streptococcus pneumoniae and the adenylate cyclase (CyaA) from Bordetella pertussis.

2. (canceled)

3. The recombinant protein according to claim 1, characterized in that the enzymatic activity of said CyaA is inactivated.

4. (canceled)

5. The recombinant protein according to claim 1 or 3, characterized in that said fragment is selected from the group consisting of:
fragments encoding a final part of the N-terminal region of PspAs of clades 2 (PspA2-F5) and 4 (PspA4-F5), wherein PspA2-F5 consists of the amino acid sequence as set forth in SEQ ID NO: 1, and PspA4-F5 consists of the amino acid sequence as set forth in SEQ ID NO: 2;
fragments encoding a final part of the N-terminal region of PspAs of clades 1 (PspA1-F5) and 3 (PspA3-F5), where PspA1-F5 consists of the amino acid sequence as set forth in SEQ ID NO: 3, and PspA3-F5 consists of the amino acid sequence as set forth in SEQ ID NO: 4;
fragments encoding a final part of the N-terminal region of PspAs from clades 2 (PspA2-F5), 4 (PspA4-F5) and 1 (PspA1-F5); and
fragments encoding a final part of the N-terminal region of PspAs from clades 2 (PspA2-F5), 4 (PspA4-F5), 1 (PspA1-F5) and 3 (PspA3-F5).

6. The recombinant protein according to any one of claims 1, 3, and 5, characterized in that said PspAs fragments are encoded by the following nucleotides sequences:
the DNA sequence as set forth in SEQ ID NO: 29 and degenerate sequences thereof that encode the PspA2-F5 as set forth in SEQ ID NO: 1;
the DNA sequence as set forth in SEQ ID NO: 30 and degenerate sequences thereof that encode the PspA4-F5 as set forth in SEQ ID NO: 2;
the DNA sequence as set forth in SEQ ID NO: 31 and degenerate sequences thereof that encode the PspA1-F5 as set forth in SEQ ID NO: 3; and/or
the DNA sequence as set forth in SEQ ID NO: 32 and degenerate sequences thereof that encode the PspA3-F5 as set forth in SEQ ID NO: 4.

7. The recombinant protein according to any one of claims 1, 3, and 5 to 6, characterized in that it comprises the following amino acid sequences:
CyaA-PspA2-F5 consisting of the amino acid sequence as set forth in SEQ ID NO: 5; or
CyaA-PspA4-F5 consisting of the amino acid sequence as set forth in SEQ ID NO: 6; or
CyaA-PspA2-F5-PspA4-F5 consisting of the amino acid sequence as set forth in SEQ ID NO: 7; or
CyaA-PspA1-F5 consisting of the amino acid sequence as set forth in SEQ ID NO: 8; or
CyaA-PspA1-F5-PspA3-F5 consisting of the amino acid sequence as set forth in SEQ ID NO: 9; or
CyaA-PspA2-F5-PspA4-F5-PspA1-F5 consisting of the amino acid sequence as set forth in SEQ ID NO: 10; or
CyaA-PspA2-F5-PspA4-F5-PspA1-F5-PspA3-F5 consisting of the amino acid sequence as set forth in SEQ ID NO: 11.

8. The recombinant protein according to any one of claims 1, 3, and 5 to 7, characterized in that it is encoded by the following nucleotide sequences:
DNA sequence as set forth in SEQ ID NO: 12 and degenerate sequences thereof that encode the recombinant protein CyaA-PspA2-F5 as set forth in SEQ ID NO: 5;
DNA sequence as set forth in SEQ ID NO: 13 and degenerate sequences thereof that encode the recombinant protein CyaA-PspA4-F5 as set forth in SEQ ID NO: 6;
DNA sequence as set forth in SEQ ID NO: 14 and degenerate sequences thereof that encode the recombinant protein CyaA-PspA2-F5-PspA4-F5 as set forth in SEQ ID NO: 7;
DNA sequence as set forth in SEQ ID NO: 15 and degenerate sequences thereof that encode the recombinant protein CyaA-PspA1-F5 as set forth in SEQ ID NO: 8;
DNA sequence as set forth in SEQ ID NO: 16 and degenerate sequences thereof that encode the recombinant protein CyaA-PspA1-F5-PspA3-F5 as set forth in SEQ ID NO: 9;
DNA sequence as set forth in SEQ ID NO: 17 and degenerate sequences thereof that encode the recombinant protein CyaA-PspA2-F5-PspA4-F5-PspA1-F5 as set forth in SEQ ID NO: 10; or
DNA sequence as set forth in SEQ ID NO: 18 and degenerate sequences thereof that encode the recombinant protein CyaA-PspA2-F5-PspA4-F5-PspA1-F5-PspA3-F5 as set forth in SEQ ID NO: 11.

9. Expression cassette characterized in that it comprises the DNA sequence selected from the group consisting of the nucleotide sequence as set forth in SEQ ID NOs: 12 to 18 and degenerate sequences thereof encoding a recombinant protein as set forth in SEQ ID NOs: 5 to 11, respectively.

10. Immunogenic composition characterized in that it comprises the recombinant protein as defined in any one of claims 1, 3, and 5 to 8, or the expression cassette as defined in claim 9, and a pharmaceutically acceptable carrier and/or adjuvant.

11. The immunogenic composition according to claim 10, for use as a vaccine.

12. The immunogenic composition according to claim 10 or 11, characterized in that it is administered parenterally or via the mucosal route.

13. The immunogenic composition according to any one of claims 10 to 12, for use in preventing infections caused by Streptococcus pneumoniae.

14. The recombinant protein as defined in any one of claims 1, 3, and 5 to 8, the expression cassette as defined in claim 9, or the immunogenic composition as defined in any one of claims 10 to 13 characterized in that it is for use as a vaccine to prevent infections caused by Streptococcus pneumoniae.

15. The recombinant protein for use, according to claim 14, characterized in that said vaccine is to prevent infections caused by Streptococcus pneumoniae that offers broad-spectrum protection against different pneumococcal isolates, regardless of serotypes.


SEQUENCE LISTING (34 sequences)

SEQ ID NO: 1 - protein, 103 aa, Streptococcus pneumoniae
SESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPG

SEQ ID NO: 2 - protein, 160 aa, Streptococcus pneumoniae
SNGEQAEQYLAAAEKDLVAKKAELAEAKIKAATKKAELEKAEAELENLLSTLDPEGKTQDELDKEAAEAELNKKVEALQNQVAELEEELSKLEDNFKDAETNNVEDYIKEGLEEAIATKKAELEKTQKELDAALNELGPDGDEEETPAPAPQPEKPAPAP

SEQ ID NO: 3 - protein, 126 aa, Streptococcus pneumoniae
LKEIDESDSEDYVKEGLRAPLQFELDVKQAKLSKLEELSDKIDELDAEIAKLEKDVEDFKNSDGEQAGQYLAAAEEDLVAKKAELEKTEADLKKAVNEPEKPAEETPAPAPKPEQPAEQPKPAPAP

SEQ ID NO: 4 - protein, 151 aa, Streptococcus pneumoniae
DGTEVIEAKLNKGEAELNAKQAELAKKQTELEKLLDSLDPEGKTQDELDKEAEEAELDKKADELQNKVADLEKEISNLEILLGGADSEDDTAALQNKLATKKAELEKTQKELDAALNELGPDGDEEETPAPAPQPEQPAPAPKPEQPAPAP

SEQ ID NO: 5 - protein, 1816 aa, synthetic construct
  REGION 1..1816: note = CyaA-PspA2-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRPASSESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPGGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 6 - protein, 1873 aa, synthetic construct
  REGION 1..1873: note = CyaA-PspA4-F5 recobinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRPASSNGEQAEQYLAAAEKDLVAKKAELAEAKIKAATKKAELEKAEAELENLLSTLDPEGKTQDELDKEAAEAELNKKVEALQNQVAELEEELSKLEDNFKDAETNNVEDYIKEGLEEAIATKKAELEKTQKELDAALNELGPDGDEEETPAPAPQPEKPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 7 - protein, 1979 aa, synthetic construct
  REGION 1..1979: note = CyaA-PspA2-F5-PspA4-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRSESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPGTRRPASSNGEQAEQYLAAAEKDLVAKKAELAEAKIKAATKKAELEKAEAELENLLSTLDPEGKTQDELDKEAAEAELNKKVEALQNQVAELEEELSKLEDNFKDAETNNVEDYIKEGLEEAIATKKAELEKTQKELDAALNELGPDGDEEETPAPAPQPEKPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 8 - protein, 1839 aa, synthetic construct
  REGION 1..1839: note = CyaA-PspA1-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRPASLKEIDESDSEDYVKEGLRAPLQFELDVKQAKLSKLEELSDKIDELDAEIAKLEKDVEDFKNSDGEQAGQYLAAAEEDLVAKKAELEKTEADLKKAVNEPEKPAEETPAPAPKPEQPAEQPKPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQVVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 9 - protein, 1993 aa, synthetic construct
  REGION 1..1993: note = CyaA-PspA1-F5-PspA3-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRPASLKEIDESDSEDYVKEGLRAPLQFELDVKQAKLSKLEELSDKIDELDAEIAKLEKDVEDFKNSDGEQAGQYLAAAEEDLVAKKAELEKTEADLKKAVNEPEKPAEETPAPAPKPEQPAEQPKPAPAPGTSDGTEVIEAKLNKGEAELNAKQAELAKKQTELEKLLDSLDPEGKTQDELDKEAEEAELDKKADELQNKVADLEKEISNLEILLGGADSEDDTAALQNKLATKKAELEKTQKELDAALNELGPDGDEEETPAPAPQPEQPAPAPKPEQPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQVVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 10 - protein, 2107 aa, synthetic construct
  REGION 1..2107: note = CyaA-PspA2-F5-PspA4-F5-PspA1-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRSESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPGTRRPASSNGEQAEQYLAAAEKDLVAKKAELAEAKIKAATKKAELEKAEAELENLLSTLDPEGKTQDELDKEAAEAELNKKVEALQNQVAELEEELSKLEDNFKDAETNNVEDYIKEGLEEAIATKKAELEKTQKELDAALNELGPDGDEEETPAPAPQPEKPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGLKEIDESDSEDYVKEGLRAPLQFELDVKQAKLSKLEELSDKIDELDAEIAKLEKDVEDFKNSDGEQAGQYLAAAEEDLVAKKAELEKTEADLKKAVNEPEKPAEETPAPAPKPEQPAEQPKPAPAPTSESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQVVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 11 - protein, 2260 aa, synthetic construct
  REGION 1..2260: note = CyaA-PspA2-F5-PspA4-F5-PspA1-F5-PspA3-F5 recombinant protein
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRSESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPGTRRPASSNGEQAEQYLAAAEKDLVAKKAELAEAKIKAATKKAELEKAEAELENLLSTLDPEGKTQDELDKEAAEAELNKKVEALQNQVAELEEELSKLEDNFKDAETNNVEDYIKEGLEEAIATKKAELEKTQKELDAALNELGPDGDEEETPAPAPQPEKPAPAPGTAASEATGGLDRERIDLLWKIARAGARSAVGTEARRQFRYDGDMNIGVITDFELEVRNALNRRAHAVGAQDVVQHGTEQNNPFPEADEKIFVVSATGLKEIDESDSEDYVKEGLRAPLQFELDVKQAKLSKLEELSDKIDELDAEIAKLEKDVEDFKNSDGEQAGQYLAAAEEDLVAKKAELEKTEADLKKAVNEPEKPAEETPAPAPKPEQPAEQPKPAPAPTSDGTEVIEAKLNKGEAELNAKQAELAKKQTELEKLLDSLDPEGKTQDELDKEAEEAELDKKADELQNKVADLEKEISNLEILLGGADSEDDTAALQNKLATKKAELEKTQKELDAALNELGPDGDEEETPAPAPQPEQPAPAPKPEQPAPAPTSESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQVVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 12 - DNA, 5448 bp, synthetic construct
  misc_feature 1..5448: note = Sequence of nucleotides that encode the CyaA-PspA2-F5
  source 1..5448: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagctctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 13 - DNA, 5622 bp, synthetic construct
  misc_feature 1..5622: note = Sequence of nucleotides that encode the CyaA-PspA4-F5
  source 1..5622: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagctcaaacggtgagcaagctgaacaataccttgctgcagctgaaaaagacttagttgctaaaaaagctgaattagcagaagctaaaattaaagctgctactaaaaaagctgaattagaaaaagcagaagctgaacttgaaaacctattatctacactagaccctgaaggtaagactcaggatgaattagataaagaagctgctgaagctgagttgaataaaaaagttgaagctcttcaaaaccaagttgctgaattagaagaagaactttcaaaacttgaagataattttaaagatgctgaaacaaacaacgttgaagactacattaaagaaggtttagaagaagctatcgcgactaaaaaagctgaattggaaaaaactcaaaaagaattagatgcagctcttaatgagttaggccctgatggagatgaagaagagactccagcgccagctcctcaaccagaaaaaccagctccagctccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 14 - DNA, 5946 bp, synthetic construct
  misc_feature 1..5946: note = Sequence of nucleotides that encode the CyaA-PspA2-F5-PspA4-F5
  source 1..5946: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagctctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactccaacgcggcggccggctagctcaaacggtgagcaagctgaacaataccttgctgcagctgaaaaagacttagttgctaaaaaagctgaattagcagaagctaaaattaaagctgctactaaaaaagctgaattagaaaaagcagaagctgaacttgaaaacctattatctacactagaccctgaaggtaagactcaggatgaattagataaagaagctgctgaagctgagttgaataaaaaagttgaagctcttcaaaaccaagttgctgaattagaagaagaactttcaaaacttgaagataattttaaagatgctgaaacaaacaacgttgaagactacattaaagaaggtttagaagaagctatcgcgactaaaaaagctgaattggaaaaaactcaaaaagaattagatgcagctcttaatgagttaggccctgatggagatgaagaagagactccagcgccagctcctcaaccagaaaaaccagctccagctccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 15 - DNA, 5520 bp, synthetic construct
  misc_feature 1..5520: note = Sequence of nucleotides that encode the CyaA-PspA1-F5
  source 1..5520: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagcctcaaagagattgatgagtctgattcagaagattatgttaaagaaggtctccgtgctcctcttcaatttgaattggatgttaagcaagctaaactatcaaaacttgaagagttgagtgataagattgatgagttagacgctgaaattgcaaaacttgaaaaagatgtagaagatttcaaaaactcagacggtgagcaagctggacaataccttgctgcagctgaagaagacttagttgctaaaaaagctgaattagaaaaaactgaagctgaccttaagaaagcagttaatgagccagaaaaaccagctgaagaaactccagctccagcaccaaaaccagagcaaccagctgaacaaccaaaaccagcgccggctcctggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 16 - DNA, 5982 bp, synthetic construct
  misc_feature 1..5982: note = Sequence of nucleotides that encode the CyaA-PspA1-F5-PspA3-F5
  source 1..5982: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagcctcaaagagattgatgagtctgattcagaagattatgttaaagaaggtctccgtgctcctcttcaatttgaattggatgttaagcaagctaaactatcaaaacttgaagagttgagtgataagattgatgagttagacgctgaaattgcaaaacttgaaaaagatgtagaagatttcaaaaactcagacggtgagcaagctggacaataccttgctgcagctgaagaagacttagttgctaaaaaagctgaattagaaaaaactgaagctgaccttaagaaagcagttaatgagccagaaaaaccagctgaagaaactccagctccagcaccaaaaccagagcaaccagctgaacaaccaaaaccagcgccggctcctggtacctcggatggcacagaagttatagaagctaaattaaacaaaggagaagctgagctaaacgctaaacaagctgagttagcaaaaaaacaaacagaacttgaaaaacttcttgacagccttgatcctgaaggtaagactcaggatgaattagataaagaagcagaagaagctgagttggataaaaaagctgatgaacttcagaataaagttgctgatttagaaaaagaaattagtaaccttgaaatattacttggaggggctgattctgaagatgatactgctgctcttcaaaataaattagctactaaaaaagctgaattggaaaaaactcaaaaagaattagatgcagctcttaatgagttaggccctgatggagatgaagaagaaactccagcgccggctcctcaaccagagcaaccagctcctgcaccaaaaccagagcaaccagctcctgcaccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 17 - DNA, 6330 bp, synthetic construct
  misc_feature 1..6330: note = Sequence of nucleotides that encode theCyaA-PspA2-F5-PspA4-F5-PspA1-F5
  source 1..6330: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagctctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactccaacgcggcggccggctagctcaaacggtgagcaagctgaacaataccttgctgcagctgaaaaagacttagttgctaaaaaagctgaattagcagaagctaaaattaaagctgctactaaaaaagctgaattagaaaaagcagaagctgaacttgaaaacctattatctacactagaccctgaaggtaagactcaggatgaattagataaagaagctgctgaagctgagttgaataaaaaagttgaagctcttcaaaaccaagttgctgaattagaagaagaactttcaaaacttgaagataattttaaagatgctgaaacaaacaacgttgaagactacattaaagaaggtttagaagaagctatcgcgactaaaaaagctgaattggaaaaaactcaaaaagaattagatgcagctcttaatgagttaggccctgatggagatgaagaagagactccagcgccagctcctcaaccagaaaaaccagctccagctccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtctcaaagagattgatgagtctgattcagaagattatgttaaagaaggtctccgtgctcctcttcaatttgaattggatgttaagcaagctaaactatcaaaacttgaagagttgagtgataagattgatgagttagacgctgaaattgcaaaacttgaaaaagatgtagaagatttcaaaaactcagacggtgagcaagctggacaataccttgctgcagctgaagaagacttagttgctaaaaaagctgaattagaaaaaactgaagctgaccttaagaaagcagttaatgagccagaaaaaccagctgaagaaactccagctccagcaccaaaaccagagcaaccagctgaacaaccaaaaccagcgccggctcctacctcggaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 18 - DNA, 6789 bp, synthetic construct
  misc_feature 1..6789: note = Sequence of nucleotides that encode theCyaA-PspA2-F5-PspA4-F5-PspA1-F5-PspA3-F5
  source 1..6789: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagctctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactccaacgcggcggccggctagctcaaacggtgagcaagctgaacaataccttgctgcagctgaaaaagacttagttgctaaaaaagctgaattagcagaagctaaaattaaagctgctactaaaaaagctgaattagaaaaagcagaagctgaacttgaaaacctattatctacactagaccctgaaggtaagactcaggatgaattagataaagaagctgctgaagctgagttgaataaaaaagttgaagctcttcaaaaccaagttgctgaattagaagaagaactttcaaaacttgaagataattttaaagatgctgaaacaaacaacgttgaagactacattaaagaaggtttagaagaagctatcgcgactaaaaaagctgaattggaaaaaactcaaaaagaattagatgcagctcttaatgagttaggccctgatggagatgaagaagagactccagcgccagctcctcaaccagaaaaaccagctccagctccaggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtctcaaagagattgatgagtctgattcagaagattatgttaaagaaggtctccgtgctcctcttcaatttgaattggatgttaagcaagctaaactatcaaaacttgaagagttgagtgataagattgatgagttagacgctgaaattgcaaaacttgaaaaagatgtagaagatttcaaaaactcagacggtgagcaagctggacaataccttgctgcagctgaagaagacttagttgctaaaaaagctgaattagaaaaaactgaagctgaccttaagaaagcagttaatgagccagaaaaaccagctgaagaaactccagctccagcaccaaaaccagagcaaccagctgaacaaccaaaaccagcgccggctcctacctcggatggcacagaagttatagaagctaaattaaacaaaggagaagctgagctaaacgctaaacaagctgagttagcaaaaaaacaaacagaacttgaaaaacttcttgacagccttgatcctgaaggtaagactcaggatgaattagataaagaagcagaagaagctgagttggataaaaaagctgatgaacttcagaataaagttgctgatttagaaaaagaaattagtaaccttgaaatattacttggaggggctgattctgaagatgatactgctgctcttcaaaataaattagctactaaaaaagctgaattggaaaaaactcaaaaagaattagatgcagctcttaatgagttaggccctgatggagatgaagaagaaactccagcgccggctcctcaaccagagcaaccagctcctgcaccaaaaccagagcaaccagctcctgcaccaacctcggaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 19 - DNA, 50 bp, synthetic construct
  misc_feature 1..50: note = Oligonucleotide sequence FORW cya-pspA2-F5
  source 1..50: mol_type = other DNA
ggcgcgtacgcgtaggccttgctagctctgaatcagaagattatgctaaa

SEQ ID NO: 20 - DNA, 49 bp, synthetic construct
  misc_feature 1..49: note = Oligonucleotide sequence REV cya-pspA2-F5
  source 1..49: mol_type = other DNA
ccgtggcctcgctggcggcggtacctggagtttctggagctggagctgg

SEQ ID NO: 21 - DNA, 45 bp, synthetic construct
  misc_feature 1..45: note = Oligonucleotide sequence FORW cya-pspA4-F5
  source 1..45: mol_type = other DNA
ggcgcgtacgcgtaggcctgctagctcaaacggtgagcaagctga

SEQ ID NO: 22 - DNA, 46 bp, synthetic construct
  misc_feature 1..46: note = Oligonucleotide sequenceREV cya-pspA4-F5
  source 1..46: mol_type = other DNA
cgtggcctcgctggcggcggtacctggagctggagctggtttttct

SEQ ID NO: 23 - DNA, 28 bp, synthetic construct
  misc_feature 1..28: note = Oligonucleotide sequence of the plasmid pAE-pspA4-F5
  source 1..28: mol_type = other DNA
ggatcctcaaacggtgagcaagctgaac

SEQ ID NO: 24 - DNA, 33 bp, synthetic construct
  misc_feature 1..33: note = REV oligonucleotide sequence of the plasmid pAE-pspA4-F5
  source 1..33: mol_type = other DNA
aagctttcatggagctggagctggtttttctgg

SEQ ID NO: 25 - protein, 1720 aa, synthetic construct
  REGION 1..1720: note = Experimental control CyaA-OVA
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADLQIDMFAIMPHLSNFRDSARSSVTSGDSVTDYLARTRRPASIINFEKLGTAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 26 - DNA, 5163 bp, synthetic construct
  misc_feature 1..5163: note = Nucleotide sequence of the experimental control CyaA-OVA
  source 1..5163: mol_type = other DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatctgcagatcgacatgttcgccattatgccgcatctgtccaacttccgcgactcggcgcgcagttcggtgaccagcggcgattcggtgaccgattacctggcgcgcacgcggcggccggctagcataattaatttcgaaaaactgggtaccgccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 27 - DNA, 49 bp, synthetic construct
  misc_feature 1..49: note = FORW oligonucleotide sequence of the plasmid A2-f51
  source 1..49: mol_type = other DNA
gaccgattacctggcgcgtacgcgccgttctgaatcagaagattatgct

SEQ ID NO: 28 - DNA, 42 bp, synthetic construct
  misc_feature 1..42: note = REV oligonucleotide sequence of the plasmid A2-f52
  source 1..42: mol_type = other DNA
tgagctagcaggcctacgcgtacctggagtttctggagctgg

SEQ ID NO: 29 - DNA, 306 bp, synthetic construct
  misc_feature 1..306: note = Nucleotide sequence encoding the fragment PspA2-F5
  source 1..306: mol_type = other DNA
tctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactcca

SEQ ID NO: 30 - DNA, 480 bp, synthetic construct
  misc_feature 1..480: note = Nucleotide sequence encoding the fragment PspA4-F5
  source 1..480: mol_type = other DNA
tcaaacggtgagcaagctgaacaataccttgctgcagctgaaaaagacttagttgctaaaaaagctgaattagcagaagctaaaattaaagctgctactaaaaaagctgaattagaaaaagcagaagctgaacttgaaaacctattatctacactagaccctgaaggtaagactcaggatgaattagataaagaagctgctgaagctgagttgaataaaaaagttgaagctcttcaaaaccaagttgctgaattagaagaagaactttcaaaacttgaagataattttaaagatgctgaaacaaacaacgttgaagactacattaaagaaggtttagaagaagctatcgcgactaaaaaagctgaattggaaaaaactcaaaaagaattagatgcagctcttaatgagttaggccctgatggagatgaagaagagactccagcgccagctcctcaaccagaaaaaccagctccagctcca

SEQ ID NO: 31 - DNA, 378 bp, synthetic construct
  misc_feature 1..378: note = Nucleotide sequence encoding the fragment PspA1-F5
  source 1..378: mol_type = other DNA
ctcaaagagattgatgagtctgattcagaagattatgttaaagaaggtctccgtgctcctcttcaatttgaattggatgttaagcaagctaaactatcaaaacttgaagagttgagtgataagattgatgagttagacgctgaaattgcaaaacttgaaaaagatgtagaagatttcaaaaactcagacggtgagcaagctggacaataccttgctgcagctgaagaagacttagttgctaaaaaagctgaattagaaaaaactgaagctgaccttaagaaagcagttaatgagccagaaaaaccagctgaagaaactccagctccagcaccaaaaccagagcaaccagctgaacaaccaaaaccagcgccggctcct

SEQ ID NO: 32 - DNA, 453 bp, synthetic construct
  misc_feature 1..453: note = Nucleotide sequence encoding the fragment PspA3-F5
  source 1..453: mol_type = other DNA
gatggcacagaagttatagaagctaaattaaacaaaggagaagctgagctaaacgctaaacaagctgagttagcaaaaaaacaaacagaacttgaaaaacttcttgacagccttgatcctgaaggtaagactcaggatgaattagataaagaagcagaagaagctgagttggataaaaaagctgatgaacttcagaataaagttgctgatttagaaaaagaaattagtaaccttgaaatattacttggaggggctgattctgaagatgatactgctgctcttcaaaataaattagctactaaaaaagctgaattggaaaaaactcaaaaagaattagatgcagctcttaatgagttaggccctgatggagatgaagaagaaactccagcgccggctcctcaaccagagcaaccagctcctgcaccaaaaccagagcaaccagctcctgcacca

SEQ ID NO: 33 - protein, 1706 aa, Bordetella pertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 34 - DNA, 5121 bp, Bordetella pertussis
  source 1..5121: mol_type = genomic DNA
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatatcgacatgttcgccattatgccgcatctgtccaacttccgcgactcggcgcgcagttcggtgaccagcggcgattcggtgaccgattacctggcgcgcacgcggcgggccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaagtggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctaaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga